(19) United States
(12) Patent Application Publication
(10) Pub. No.: US 2015/0073285 A1
(43) Pub. Date: Mar. 12, 2015

(54) UNIVERSAL ECG ELECTRODE MODULE FOR SMARTPHONE

(71) Applicant: AliveCor, Inc., San Francisco, CA (US)

(72) Inventors: David E. ALBERT, San Francisco, CA (US); Bruce Richard SATCHWELL, San Francisco, CA (US)

(21) Appl. No.: 14/479,105

(22) Filed: Sep. 5, 2014

Related U.S. Application Data
(60) Provisional application No. 61/874,806, filed on Sep. 6, 2013.

(51) Int. Cl. A61B 5/00; H01R 24/28; H04B 5/00

(52) U.S. Cl. CPC A61B 5/0022; A61B 5/0006; A61B 5/6898; H04B 5/0031; H01R 24/28
USPC 600/509

(57) ABSTRACT

Apparatuses and methods for sensing an electrocardiogram (ECG) of a subject using an ECG module that may removably connect to a mobile telecommunications device through a base unit, and which may further then wirelessly communicate with the mobile telecommunications device.

### CROSS REFERENCE

This utility application claims benefit of U.S. provisional application 61/874,806, filed on Sep. 6, 2013, which is incorporated herein by reference. The subject matter of this application is further related to the subject matter of U.S. patent application Ser. No. 13/752,048, filed Jan. 28, 2013 and titled “ULTRASONIC TRANSMISSION OF SIGNALS,” Publication No. US-2013-0197320-A1; U.S. patent application Ser. No. 13/969,446, filed Aug. 16, 2013 and titled “ULTRASONIC TRANSMISSION OF SIGNALS;” U.S. patent application Ser. No. 13/964,490, filed Aug. 12, 2013 and titled “HEART MONITORING SYSTEM USABLE WITH A SMARTPHONE OR COMPUTER;” and U.S. patent application Ser. No. 13/108,738, filed May 16, 2011 and titled “WIRELESS, ULTRASONIC PERSONAL HEALTH MONITORING SYSTEM,” Publication No. US-2011-0301439-A1, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This patent application describes apparatuses, including systems, software (stored or located as a non-transient medium), and devices, as well as methods (including methods for manufacturing and methods of using these apparatuses) for taking electrocardiogram (ECG) information from a subject. In particular, described herein are universal ECG acquisition devices that are compatible with a variety of mobile telecommunications devices.

### BACKGROUND

Electrocardiogram (ECG) monitoring is often highly useful in maintaining optimal cardiac function in both healthy and non-healthy subjects. Such monitoring can generate data that can be used by the subject as well as healthcare professionals to guide preventative and therapeutic treatment decision making.

Described herein are ECG-sensing and transmitting apparatuses for use with mobile telecommunications devices for detecting ECGs.

### SUMMARY OF THE DISCLOSURE

In general, the apparatuses (including devices and systems) and methods described herein are for use in detecting biological signals such as electrocardiograms (ECGs). In particular, described herein are ECG sensing devices for use with mobile telecommunications devices such as smartphones, tablet computers, smart watches, wearable computing devices, and the like so that the mobile telecommunications device may receive biological signals measured directly from a patient.

The apparatuses described herein may be referred to as “universal” or as including a “universal module” or “universal adapter” because they may be used with any mobile telecommunications device, regardless of the form factor of the mobile telecommunications device. For example, the apparatuses described herein may include a base unit that may be configured to engage directly with the mobile telecommunications device and a separate ECG electrode module (“sensing module”, “ECG module”, “electrode module”) that engages with the base unit regardless of the form factor of the mobile telecommunications device to which the base unit is attached.

For example, an electrocardiogram (ECG) sensing device for use with a mobile computing device can comprise a base unit; a universal coupler; and an ECG module that removably couples to the base unit as well as other base units via the universal coupler. The ECG module can further comprise a conductive contact surface for contacting a skin surface of a subject and generating a signal in response and a transmitter for wirelessly transmitting the signal to the computing device. The signal transmitted can comprise for example any of a radio signal, a microwave signal a visible light signal, an infrared signal, a sonic signal, an ultrasonic signal, an electromagnetic induction signal, a WiFi signal, a ZigBee signal, a Bluetooth signal, a Bluetooth LE signal, or a wireless signal.

The universal coupler can comprise an opening through the base unit sized such that said ECG module may extend partially therethrough, said opening having an edge region configured to engage a periphery of said ECG module.

In addition to the two-part configuration of the base unit and ECG electrode module, the apparatuses described herein may be universally used with a large variety of mobile telecommunications devices because they transmit data to the mobile telecommunications device wirelessly, even though the mobile telecommunications device is connected to base unit. Thus, the ECG electrode module does not directly couple with the mobile telecommunications device, but is held with, and connected to, the mobile telecommunications device only through the base unit. In general, although the ECG electrode module may receive, record, and/or analyze ECG signals from a patient, it may operate in conjunction with the mobile telecommunications device (including client software/hardware/firmware in/on the mobile telecommunications device) to analyze, display, store and/or transmit the received ECG signals or information about them. In particular, the ultrasonic variations of the apparatuses described herein, which may configure sensed ECG signals for ultrasonic transmission by the ECG electrode module to the mobile telecommunications device, may be considered “universal” because they use the microphone built into the mobile telecommunications device.

Also described herein are ECG electrode modules that are adapted so that they can be set onto a conductive surface face-down (e.g., so that the electrodes face the conductive surface), and the electrode surfaces will not contact the conductive surface, which may otherwise interfere with the power consumption of the device (e.g., draining the battery). For example, in some variations, the devices include a rest or resting surface or surfaces (or “foot”) that projects above the height of the electrodes.

The base unit may typically include one or more engagement surfaces (mobile telecommunications device engagement surface) configured to engage with the mobile telecommunications device (e.g., smartphone, tablet, etc.). The base unit may also include an ECG electrode module engagement region (module engagement region) for connecting to an ECG electrode module. The mobile telecommunications device engagement surface may be an adhesive contact surface that may be stuck onto the mobile telecommunications device. In some variations the base unit is configured as, or includes, a case for the mobile telecommunications device. The case may include an inner region within which the mobile telecommunications device may sit, and may have one or more transparent regions, for example, for viewing a screen of the mobile telecommunications device, and/or openings, e.g., for any controls (buttons, knobs, switches, etc.) or plug-in regions of the mobile telecommunications device. The mobile telecommunications device engagement surface or region may correspond to the pocket formed by the case into which the mobile telecommunications device is held. An ECG electrode module engagement region may be located on back of the case embodiment; in some variations the back of the case embodiment may be formed to include the ECG module engagement region. In some variations, the ECG module region includes a lip or edge for retaining the ECG electrode module. In some variation the base unit does not include a case, but may be adhesively secured to a case (or directly to the back of the phone). In some variations, different base units may be configured as a variety of different cases that each fit a different size of mobile telecommunications device (e.g., mobile telecommunications device having different form factors); but each of the different form-factor base units may fit the same size ECG electrode module.

In general, an ECG electrode module includes two (or more) electrodes that may be contacted by or to a subject to take an ECG reading from the subject. The ECG module may be at least partially enclosed in a housing, with the electrodes (e.g., a first electrode and a second electrode) on the outer surface of the housing. The electrodes may together be referred to as an electrode assembly. The ECG electrode module housing may enclose the hardware, software, and/or firmware of the module, including, for example, a converter for converting an ECG signal detected from the subject touching the electrodes into a transmission signal for transmission by the module to the mobile telecommunications device. The housing may also include any signal conditioning components helpful for sensing and processing an ECG signal. For example, the housing may include (e.g., as part of the electrode assembly and/or as part of a controller, processor and/or converter), amplifiers, filters, or the like. The converter may include an oscillator or the like (e.g., for generating one or more carrier frequencies). The housing may also at least partially enclose one or more transmitters for emitting or broadcasting the wireless signal to be received by the mobile telecommunications device. A transmitter may include a sound transmitter (e.g., ultrasound transducer such as a piezo, etc.), a radio transmitter, a magnetic inductive transmitter, or any other appropriate transmitter/emitter for broadcasting a wireless signal.

For example, described herein are universal electrocardiographic (ECG) detection apparatuses for use with a mobile telecommunications device, the apparatus comprising: a base unit configured to couple to the mobile telecommunications device; and an ECG electrode module that engages with the base unit, the ECG module further comprising: a housing having a bottom side that engages with the base unit; an electrode assembly on a top side of the housing, opposite the bottom side, the electrode assembly comprising a first electrode and a second electrode; a converter assembly within the housing and configured to receive an ECG signal from the electrode assembly and to output a transmission signal comprising the ECG signal; and a transmitter within the housing configured to wirelessly broadcast the transmission signal from the ECG module for receipt by the mobile telecommunications device; wherein the ECG module releasably locks into the base unit and can be released from the base unit.

In general, the base unit may be removably connected to the ECG module. For example, the ECG module may be snap-fit into the base unit, the ECG module may be friction fit into the base unit, the ECG module may be screwed into the base unit, the ECG module may be clamped into the base unit, etc. The base unit may include at least one lip region on an upper surface that is configured to engage the ECG module, such as an edge, rim or corresponding lip region on the ECG module. In some variations the base unit and/or ECG module may include a lock, latch or the like the hold the two components together until they are manually released.

The base unit may be connected to a mobile telecommunications device by any appropriate manner. For example, in some variations the base unit is adhesively attached to a mobile telecommunications device or a case for a mobile telecommunications device. Thus, in some variations, the base unit comprises an adhesive backing.

In some variations the base unit is configured to hold a mobile telecommunications device. For example, the base unit may be configured as a case, e.g., a smartphone case. The base unit may include a cradle region for releasably securing to the ECG module. In some variations the base unit configured as a case includes an opening that is sized to fit a portion of the ECG module (e.g., the front portion) through the case, while the back region remains within the case; when a mobile telecommunications device is inserted into the case, the base unit may hold the ECG module to the mobile telecommunications device. The base unit may include an opening through the case that is sized so that ECG module may extend partially through the opening; the opening having an edge region configured to engage an edge region on the ECG module. For example, the opening through the base unit (e.g., case) may be slightly undersized relative to the size of the ECG module, so that a lip, edge, rim, and/or peripheral region of the ECG module may be held on one side of the opening while the electrodes and any other portion of the ECG module projects out of the case and from the back of the case and any mobile telecommunications device within the case.

As mentioned, the base unit may include any attachment region and mechanism. For example, the base unit may include a frictional engagement region configured to releasable engage the ECG module.

The ECG module may be configured as an elongate and/or circular and/or rectangular shape. In one example the ECG module is oval. In general, the ECG module may include a pair of electrodes that are each configured to be separately touched one of the subject's hands (right hand and left hand) simultaneously. For example, a right arm, left arm (by touching the right hand to the first electrode and the left hand to the second electrode (or vice versa) may result in a single lead (e.g., Lead I) measurement.

Any of the variations described herein may also be configured so that the electrode(s) do not contact the surface of a table when the apparatus is placed electrodes down onto the surface. For example, the ECG module may further list a rest surface projecting from the top side of the housing above the first and second electrodes, so that the first and second electrodes do not contact a table surface when the module is placed on the table surface with the top side facing the table surface.

For example, an ECG sensing system can comprise a mobile computing device; and an ECG module comprising a conductive contact surface for contacting a skin surface of a subject; and an elevated portion having a height that projects above the height of the conductive contact surface.

In general, the size of the ECG module in any of the variations described herein may be configured to be thin (e.g., slim) and dimensioned to readily fit onto a mobile telecommunications device such as a smartphone without increasing the overall size (and weight) of the mobile telecommunications device too much. For example, the ECG module may be thin (e.g., less than 0.5 inches, but preferably less than about 0.4 inches, less than about 0.3 inches, less than about 0.2 inches, etc.). In some variations the ECG module is between about 0.1 inches and about 0.5 inches thick. The thickness of the ECG module may refer to the maximum thickness or height (e.g., the thickness of the module at the region of a rest surface, if one is present in a particular embodiment) of the module. The overall thickness of the module may vary across the length and width of the module; for example, the profile of the outer surface of the module (including the electrodes) may be curved and/or tiered so that different regions have different heights. The edge regions of the module may be lower (smaller) than the more central regions of the module, so that the edges taper down to a thinner profile. In some variations, the outer edge region may include a lip or rim to help retain the module in the base unit.

In the plane of the ECG module, the module may have a shape that is configured to mate with the base unit, and is configured to be attached to a mobile telecommunications device without extending beyond the footprint of most mobile telecommunications devices or interfering with elements already on the mobile telecommunications device, such as cameras, controls, or the like. The ECG modules may also be sufficiently large so that the electrode(s) may be easily contacted by the subject's hands (fingers, thumb, etc.) to reliably take an ECG reading. For example, the ECG module may be between about 1 inch and about 6 inches long, and between about 0.5 inch and about 4 inches wide. More particularly, the length of the ECG module may be between about 1 inches and about 5 inches long, between about 1 inch and about 4 inches long, or between about 1 inches and about 3 inches long. More particularly, the width of the ECG module may be between about 0.5 inches and about 3 inches wide, between about 0.5 inches and 2 inches, between about 0.5 inches and about 1 inch wide, or between about 1 inch and about 3 inches wide, or between about 1 inches and about 2 inches wide.

In general the ECG module may be planar, so that the width and length are larger (or much larger) than the height/thickness. The overall shape of the ECG electrode module in the plane of the module may be any appropriate shape, including symmetric (e.g., circle, square, rectangle, oval, barbell, etc.) and asymmetric shapes.

As mentioned, in any of these variations, the ECG module may have a rim, lip, threading, or the like region that is configured to engage with a lip on the base unit. The rim may be along all or a portion (or portions) of the outer perimeter of the module.

In general the electrode assembly may include at least a first and second electrode that are configured to be contacted (held, touched, etc.) by a left hand a right hand, respectively, of a subject holding the mobile telecommunications device to which the module is attached. The electrodes may be any conductive material, and may be “dry” (e.g., bare metal or other conductor) or may be used with a conductive material (paste, gel, etc.). Typically, the electrodes may be configured to provide accurate ECG readings when used dry, and contacting the subject's skin (fingers, hands, etc.).

The housing of the ECG module may hold (or at least partially enclose) any appropriate electronics useful to detect, process (e.g., amplify, filter, etc.) and transmit (e.g., as ultrasound in some variations) the ECG signals and/or information about detected ECG signals. For example, the apparatus may include a converter that is configured to convert sensed ECG signals into a transmission signal appropriate for transmission by a transmitter within the ECG module housing. The converter may also include filters, amplifiers and may convert analog signals to digital signals, and/or may operate entirely in analog, or may operate in both analog and digital regimes. A converter may include one or more oscillators for modulating a sensed ECG signal (adding a carrier frequency/frequencies, amplitude modulation, frequency modulation, etc.) in preparing the transmission signal. In implementing a converter, the converter may include or be formed of a processor (e.g., microprocessor) or to other circuitry, including general or specialized/dedicated circuitry.

Of particular, but not exclusive, interest herein are converters configured to prepare sensed ECG signals for transmission as an audio (e.g., ultrasound) signal by an ultrasound transmitter in the ECG electrode module. For example, a converter may be configured to output a transmission signal as an ultrasound transmission signal encoding the ECG signal for transmission as an ultrasound signal having a frequency of 17 kHz or greater. The signal maybe encoded as a digital (e.g., FSK) ultrasound signal, an analog (e.g., amplitude and/or frequency modulated signal) or a hybrid digital and analog signal (see, e.g., U.S. patent application Ser. No. 13/969,446, previously incorporated by reference in its entirety).

The ECG module may also include any appropriate transmitter for wirelessly transmitting the transmission signal prepared by the converter (or in some variations, directly transmitting the sensed ECG signal). As used herein, wireless transmission of a signal includes any appropriate wireless modality, including, but not limited to a radio signal, a microwave signal a visible light signal, an infrared signal, a sonic signal, an ultrasonic signal, an electromagnetic induction signal, a WiFi signal, a ZigBee signal, a Bluetooth signal, a Bluetooth LE signal, or a wireless signal.

Further, the transmitter may paired with the converter, which may be configured to convert the ECG signal(s) and/or information about the ECG signal(s) into transmission signals for broadcast as a radio signal, a microwave signal, visible light signal, an infrared signal, a sonic signal, an ultrasonic signal, and/or an electromagnetic induction signal.

For example, in some variations, the transmitter is an ultrasound transmitter, configured to output the transmission signal as an ultrasound signal for detection by a microphone of the mobile telecommunications device.

As mentioned, the ECG module typically releasably connects to the base unit. For example, the ECG module may releasably snap fit into the base unit.

Another example of an apparatus as described herein is an electrocardiographic (ECG) detection apparatus for use with a mobile telecommunications device, the apparatus comprising: a base unit configured to couple to the mobile telecommunications device; and an ECG electrode module configured to removably engage with the base unit via a universal coupler, the ECG module further comprising: a housing having a bottom side configured to removably engage with the base unit; an electrode assembly on a top side of the housing, opposite the bottom side, the electrode assembly comprising a first electrode and a second electrode; a rest surface projecting from the top side of the housing above the first and second electrodes, so that the first and second electrodes do not contact a table surface when the module is placed on the table surface with the top side facing the table surface; a converter assembly within the housing and configured to receive an ECG signal from the electrode assembly and to output a transmission signal comprising the ECG signal; and a transmitter within the housing configured to wirelessly transmit the transmission signal from the ECG module to the mobile telecommunications device; wherein the ECG module releasably locks into the base unit and can be released from the base unit. Any of the features of the other embodiments described herein may be incorporated into this type of apparatus.

Another example of an electrocardiographic (ECG) detection apparatus for use with a mobile telecommunications device includes: a case configured to hold the mobile telecommunications device, the case comprising a base unit configured to couple to the mobile telecommunications device; and an ECG electrode module configured to removably engage with the base unit via a universal coupler, the ECG module further comprising: a housing having a bottom side, the bottom side comprising a rim region configured to removably engage with the base unit; an electrode assembly on a top side of the housing, opposite the bottom side, the electrode assembly comprising a first electrode and a second electrode; a converter assembly within the housing and configured to receive an ECG signal from the electrode assembly and to output a transmission signal comprising the ECG signal; and a transmitter within the housing configured to wirelessly transmit the transmission signal from the ECG module to the mobile telecommunications device; wherein the ECG module releasably locks into the base unit and can be released from the base unit by a user. Any of the features of the other embodiments described herein may be incorporated into this type of apparatus.

Another example of an electrocardiographic (ECG) detection apparatus for use with a mobile telecommunications device includes: a base unit configured to couple to the mobile telecommunications device; and an ECG electrode module configured to removably engage with the base unit via a universal coupler. The ECG module further includes: a housing having a bottom side configured to removably engage with the base unit; an electrode assembly on a top side of the housing, opposite the bottom side, the electrode assembly comprising a first electrode and a second electrode; a converter assembly within the housing and configured to receive an ECG signal from the electrode assembly and to output a signal comprising the ECG signal for transmission as an ultrasound signal having a frequency of 17 kHz or greater; and an audio transmitter within the housing configured to output the ultrasound signal from the ECG module for detection by a microphone of the mobile telecommunications device; wherein the ECG module releasably locks into the base unit and can be released from the base unit. Any of the features of the other embodiments described herein may be incorporated into this type of apparatus.

Also described herein are methods of coupling a universal electrocardiographic (ECG) detection apparatus to a mobile telecommunications device. In general, these methods may include attaching the base unit to the mobile telecommunications device (e.g., smartphone) in any appropriate manner, and coupling the ECG electrode module to the base unit. The ECG module may be releasably coupled by a universal coupler that is for example a snap fit coupler, or a friction fit coupler, or clamping the two together via a coupler, etc., including any appropriate method of releasably connecting the two. A lock or locking mechanism may be used to hold the ECG module to the base; the lock/locking mechanism may later be released to separate the ECG module from the base unit, allowing replacement of a battery within the ECG module, or replacement of all or other components of the ECG module. The base unit may be permanently or temporarily attached to the mobile telecommunications device. For example, the base unit may be adhesively attached to the smartphone and/or a case for a smartphone. In some variations, the base unit is configured as a case for a smartphone; the case may include a cradle or other ECG module engagement region (which may include a lock/locking mechanism) for holding the ECG module.

For example, in some variations the method of coupling an ECG electrode module (universal module) to a mobile telecommunications device such as a smartphone comprises: coupling a base unit to the mobile telecommunications device; releasably attaching an ECG module to the base unit, the ECG module comprising a housing, an electrode assembly on a top side of the housing, a converter assembly within the housing, and a transmitter within the housing; receiving an ECG signal from the electrode assembly; converting the ECG signal using the converter assembly into a transmission signal configured for wireless transmission; broadcasting the transmission signal from the ECG module; and receiving the transmission signal by the mobile telecommunications device.

In general, the ECG signal may be converted into a transmission signal configured for any appropriate wireless transmission. For example, the step of converting the ECG signal may comprise converting the ECG signal using the converter assembly into a transmission signal configured for transmission as an ultrasound signal having a frequency of 17 kHz or greater. Similarly, the step of broadcasting (transmitting or wirelessly transmitting) may include broadcasting the transmission signal using any appropriate modality. For example, broadcasting may include broadcasting the transmission signal from the ECG module comprises emitting the transmission signal from the ECG module as an ultrasound signal.

As mentioned above, any appropriate transmitter may be used. In variations in which the wireless transmitter is an ultrasound transmitter, the transmitter may use inaudible ultrasound (e.g., >10 kHz, >12 kHz, >15 kHz, >18 kHz, >19 kHz) that can be received by a microphone on the mobile telecommunications device and transmitted and/or further processed by the mobile telecommunications device. Examples of such systems are described in U.S. Pat. No. 8,301,232, and U.S. Patent Application Publication Nos.: US-2011-0301435 and US-2011-0301439, and by PCT Application No. PCT/US2013/023370, now Publication No. WO 2013/112979, each of which is herein incorporated by reference in its entirety.

The step of converting the ECG signal may comprise converting the ECG signal using the converter assembly into a transmission signal configured for transmission as one or more of: a radio signal, a microwave signal, visible light signal, an infrared signal, a sonic signal, an ultrasonic signal, and an electromagnetic induction signal.

In some variations, the base unit is directly coupled to the mobile telecommunications device. For example, the base unit may be adhesively secured to a smartphone; in some variations the base unit may be adhesively secured to a phone case. Coupling a base unit to the mobile telecommunications device may comprise placing a mobile telecommunications device within a case coupled with or forming part of the base unit. Although most of the variations described herein include the use of an intermediate base unit to couple the ECG module to the phone, in some variations it may be desirable to couple the ECG module (e.g., via an adhesive backing) directly to a mobile telecommunications device (e.g., smartphone) or case for a mobile telecommunications device. In some variations, and particularly those using an adhesive backing on the ECG module, the device may be configured so that a battery maybe changed from a top/side surface rather than the back.

The order in which the base unit is coupled to the mobile telecommunications device and the ECG module is coupled to the base unit may depend on the configuration of the base unit. In some variations coupling a base unit to the mobile telecommunications device may comprises coupling the base unit after releasably attaching the ECG module to the base unit. In some variations coupling a base unit to the mobile telecommunications device may comprises coupling the base unit before releasably attaching the ECG module to the base unit. In some variations, the order does not matter, and coupling a base unit to the mobile telecommunications device may comprises coupling the base unit either before or after releasably attaching the ECG module to the base unit.

As mentioned above, the base unit may be coupled with the ECG module in any appropriate manner, particularly releasably securing the ECG module to/in the base unit. For example, coupling the base unit may comprise snapping the ECG module into the base unit.

In one example of a method of coupling a universal electrocardiographic (ECG) detection apparatus to a mobile telecommunications device, the method may include the steps of: coupling a base unit to the mobile telecommunications device; releasably attaching an ECG module to the base unit, the ECG module comprising a housing, an electrode assembly on a top side of the housing, a converter assembly within the housing, and an audio transmitter within the housing; receiving an ECG signal from the electrode assembly; converting the ECG signal using the converter assembly into a transmission signal configured for transmission as an ultrasound signal having a frequency of 17 kHz or greater; emitting the ultrasound signal from the ECG module; and receiving the ultrasound signal by the mobile telecommunications device.

In general, a patient (as used herein) may be a human or non-human patient, including, but not limited to animals (dogs, cats, horses, etc.). Thus any of the apparatuses or methods described herein may be used for veterinary use or configured as veterinary products.

In general, a mobile telecommunications device may include any mobile telecommunications device such as, but not limited to, smartphones or other mobile (e.g., cellular) phone or equivalent, including an iPhone, Droid or the like. A mobile telecommunications device typically may include a processor or other computing module/device which may run software, hardware of the like, including machine readable code configured to operate the device to receive and/or send information from the apparatus described herein. Such code may be provided with, or separately from, the apparatus described. Mobile telecommunications device may be referred to (and includes) a cell or cellular phone or telephone, a mobile phone or telephone, a smartphone, an handheld computer, tablet, a wearable computer, or the like. Code may be referred to a software, or application software (“app” or “application”) and may be downloaded from a remote location onto the mobile telecommunications device. Thus, also described herein are non-transitory, computer-readable storage mediums/media storing a set (or sets) of instructions capable of being executed by a mobile telecommunications device, that, when executed by the mobile telecommunications device, causes the mobile telecommunications device to operate as described herein. For example, described herein are a non-transitory computer-readable storage medium storing a set of instructions capable of being executed by a smartphone, that, when executed by the smartphone, causes the smartphone to receive signals (e.g., radio, optical, sonic, ultrasonic, etc.) from encoding ECG data from the ECG electrode module. This set of instructions (“logic”, “control logic”, or software) may further be configured to cause the smartphone to analyze (filter, smooth, average, detect features) and/or store and/or transmit the ECG data including, but not limited to the ECG trace and/or extracted information from the ECG trace. The set of instructions may also be configured to determine additional information about the subject from whom the ECG was recorded.

As mentioned, when the base unit is configured as a case or for use on a case, the case may be configured to hold a mobile telecommunications device within a cavity, or to otherwise be applied over the mobile telecommunications device. The case may therefore include an inner surface or surfaces for holding the mobile telecommunications device, and may have a front region through which the screen and/or any controls of the mobile telecommunications device may be seen and/or manipulated. For example, the case may include a cut-out region or a transparent covering though which the mobile telecommunications device may be seen. A cradle or other ECG module engagement region may be mounted on the outer surface (e.g., a back surface) of the case. The case may also include one or more other openings for accessing controls, inputs, outputs, or connection regions (e.g., jacks, plug-in receptacles, etc.) of the mobile telecommunications device.

The electrode assembly of some of the ECG modules described herein may be configured to include wires and electrodes that extend from the housing of the ECG module. For example, in some variations, the ECG module includes first and second wires on and extending from the top side of the housing with electrode contacts at the ends of the wires. The electrode contacts may be attached to a subject/patient. The footprint of the ECG module, e.g., the size and shape of the bottom side of the housing, may be configured identically to the ECG modules having electrodes forming part of the surface of the housing, so that both types of ECG modules may fit into the same base unit.

As will be illustrated below, the ECG module may be configured so that the electrodes do not contact a table surface when the apparatus is placed on the table with the electrodes (electrode assembly) facing the table. This permits the device to be placed down on a metal surface, as is often found in hospital or other medical settings, without creating a conductive pathway between the electrodes and thereby potentially discharging (and/or draining power from the apparatus). In some variations, the electrodes are recessed relative to the outer surface of the ECG module. For example, the electrodes may be recessed within a material forming the housing of the ECG module. Alternatively or additionally, the ECG module may include one or more projections on which the case may rest when placed back-surface down, preventing one or more electrodes from contacting the surface. For example, the outer surface of the ECG module may include one or more rest surfaces or spacers that extend at least a portion of the outer surface relative to the outer contact surfaces of the electrode surfaces so that the outer contact surfaces of the electrodes are recessed relative to an outer surface of the one or more spacers. In general, a rest surface (which may also be called a spacer) may refer to one or more projections of the outer (front) surface of the ECG module having a height (above the base unit) greater than the height of the electrode(s). For example, a rest surface may be a bump, island, bar, piece, tab, etc., extending from the back surface, in some variations around (e.g., all or partially surrounding) the electrodes.

In general, the electrodes of the electrode assembly may be of sufficient surface area for easily making reliable contact with the patient's hands and/or other body part (leg, chest, etc.). The electrodes may be of different shapes and/or sizes, or they may be the same shape and/or size. In some variations, the surface areas of the electrodes are approximately the same.

In another variation, an ECG device comprises a base unit and an ECG module coupled to the base unit, wherein the ECG module comprises a conductive contact surface for contacting a skin surface of a subject, and a transmitter for wirelessly transmitting a signal.

The base unit can couple with a mobile computing device. The base unit can removably couple with the mobile computing device and the ECG module can removably couple with the base unit.

The base unit can comprise an opening through the case that is sized so that an ECG module may extend partially through the opening; the opening having an edge region configured to engage an edge region on the ECG module.

The conductive contact surface can comprise a sensor that reads data from the skin surface of a subject. Further, the signal transmitted from the ECG module can comprise the data from the sensor. The conductive contact surface comprises an ECG electrode.

The signal transmitted from the ECG sensing module can comprise one or more of a radio signal, a microwave signal, visible light signal, an infrared signal, a sonic signal, an ultrasonic signal, and an electromagnetic induction signal.

The ECG module can be between about 1 inch and about 5 inches long, and between about 1 inch and about 3 inches wide, and between about 0.1 inches to about 0.5 inches thick.

In another variation, an ECG system can comprise a mobile computing device; a base unit removably coupled to the mobile computing device, and an ECG module coupled to the base unit, the ECG module comprising a conductive contact surface for contacting a skin surface of a subject; and a transmitter for wirelessly transmitting a signal.

The computer device can comprise a display and a wireless receiver that receives the signal transmitted by the sensing module, the transmitted signal comprising an ECG. The display can comprise a graphic or text representation of an ECG.

In another variation, a method for sensing an ECG of a user, the method comprising providing a base unit and an ECG module that couples to the base unit; sensing the ECG of the user with a portion of the ECG module at least partially contacted with a skin of the user; transmitting wirelessly the sensed ECG to a mobile computing device coupled to the base unit; and displaying the ECG on a display portion of the mobile computing device.

Another aspect of the present disclosure describes an electrocardiogram (ECG) sensing device for use with a mobile computing device, said ECG sensing device comprising a base unit; a universal coupler; and an ECG module removably and universally couplable to said base unit via said universal coupler.

Another aspect of the present disclosure describes a method of sensing an ECG of a user which may comprise coupling removably an ECG module with a base unit removably coupled to a mobile computing device; sensing the ECG of the user with the ECG module at least partially contacted with a skin surface of the user; transmitting wirelessly the sensed ECG to the mobile computing device; displaying the ECG on a display of the mobile computing device; and decoupling the ECG module from the base unit.

### BRIEF DESCRIPTION OF THE DRAWINGS

FIG. 1 is a pictorial representation of a body showing an example of the electrode placement for taking a standard 12-lead ECG.

FIG. 2A shows a front view of one variation of an integrated ECG sensing/transmitting apparatus that does not include a separate base/ECG module; the electrodes are integrated into the housing of the case/cover for the mobile telecommunications device. In this example, a smartphone is shown inserted into the integrated case and ECG module.

FIGS. 2B, 2C and 2D show left side, back, and right side views, respectively, of the apparatus of FIG. 2A.

FIGS. 3A and 3B show back perspective and back views, respectively, of an integrated ECG sensing/transmitting apparatus similar to the variation shown in FIG. 2A, but including projections on the surface of the electrodes forming a rest surface as described herein.

FIGS. 4A-4C show one variation of an ECG module (ECG electrode module or universal module) configured to wirelessly communicate ECG data to a smartphone in top, side perspective and bottom perspective views, respectively.

FIG. 5A shows an ECG module similar to the one shown in FIGS. 4A-4C as a top perspective view. FIG. 5B shows a top view of the same ECG module, including exemplary dimensions (in mm).

FIG. 6 is a circuit diagram of one variation of an ECG module as described herein.

FIG. 7A is a side perspective view of an ECG module releasably engaged with a base unit. FIG. 7B shows the ECG module of FIG. 7A docking with a base unit.

FIGS. 8A-8C show top, front side and left side perspective views, respectively, of an ECG module such as the one shown in FIGS. 4A-5B, including exemplary dimensions (in mm) held within a base unit.

FIGS. 9A and 9B show bottom and side views, respectively, of an ECG module such as the one shown in FIGS. 4A-5B, held within a base unit.

FIG. 9B shows a cross-section through an exemplary side view (e.g., left side view) of the apparatus (ECG module and base unit) shown in FIG. 9A.

FIG. 10 is an exploded view of the ECG module and base unit of FIGS. 9A-9B.

FIGS. 11A and 11B show back and back perspective views, respectively, of a base unit forming (or attached to) a case for a smartphone to which an ECG module may be coupled.

FIG. 12 shows a perspective view of a base unit configured as a smartphone case to which an ECG module has been coupled.

FIG. 13A shows a top perspective (elevational) view of one variation of a base unit for releasably coupling to an ECG module.

FIGS. 13B, 13C, 13D, 13E, 13F and 13G show top, front side, left side, right side, back side and bottom views, respectively of the base unit of FIG. 13A.

FIG. 14 show another example of a base unit with exemplary dimensions indicated for illustrative purposes only.

FIG. 15A shows a top perspective view of another variation of a base unit, configured as a smartphone case, for use with an ECG module.

FIGS. 15B and 15C show front side perspective and bottom perspective views, respectively of the base unit of FIG. 15A.

FIGS. 16A-16C illustrate another variation of an apparatus including a base unit and an ECG module, as described herein, from the left side, back (top), and right side views, respectively. The base unit in this example is configured as an ECG case having an opening into which the ECG electrode module is secured.

FIGS. 17A, 17B and 17C show top, side perspective (elevational) and front views, respectively of another variation of an ECG module including plugs for electrodes on the top surface of the ECG module.

FIG. 18A shows a top perspective view of one variation an ECG module similar to the variation shown in FIG. 17A-17C.

FIGS. 18B, 18C, 18D, 18E, 18F and 18G show top, front side, left side, right side, back side and bottom views, respectively of the base unit of FIG. 18A.

FIG. 19 is a schematic representation of a method for sensing an ECG of a subject.

FIG. 20 is a schematic representation of an exploded view of an ECG sensing system.

FIGS. 21A, 21B, and 21C show an exploded view, top view, and side view, respectively of a slim ECG module.

### DETAILED DESCRIPTION

In general, described herein are apparatuses and methods for generating an electrocardiogram (ECG) from a patient with a universal ECG electrode module that is attached to a mobile telecommunications device through a base unit but that communicates wirelessly with the mobile telecommunications device. The base unit may be specifically attached to mobile telecommunications devices of different form factors (e.g., sizes and shapes), while the ECG module may have a single form factor that can therefore be used with any of these mobile differently shaped/sized telecommunications devices. This base unit may therefore act as an adapter attaching the ECG electrode module to a variety of differently shaped/sized mobile telecommunications devices. The ECG electrode module may therefore be referred to as universal, e.g., able to be used across the ‘universe’ of different form factor mobile telecommunications devices.

Also described herein are methods of connecting a universal ECG electrode module to a mobile telecommunications device using a base unit, and methods of using the universal ECG module to detect and transmit ECG data.

In general, an ECG detection apparatus for use with a mobile telecommunications device may include a base unit configured to couple to a mobile telecommunications device and an ECG electrode module configured to engage with the base unit. The ECG detection apparatus does not include the mobile telecommunications device (and may be provided separately). The base unit may removably/releasably engage the ECG detection apparatus. For example, in some variations, the ECG detection apparatus may be secured into the base unit and later released from the base unit, and attached to another, different base unit, or attached back to the same base unit. The removable attachment may include a lock, latch, or any other appropriate securement for holding the ECG electrode module to the base unit until it is manually (and/or automatically) released; until release the ECG module and base unit may be securely held together. In some variations the ECG module is secured to the base unit while a mobile telecommunications device is coupled to the base unit; the ECG electrode module is released when the mobile telecommunications device is uncoupled from the ECG base unit.

A mobile telecommunications device may include any appropriate wireless telecommunications device, including smartphones (e.g., iPhone, Android, etc.), tablet (iPad, etc.), laptop, PDA, wearables, etc. In some variations the base unit is configured as a case and/or attachment to the mobile telecommunications device. The ECG electrode module portion of the apparatus may communicate information wirelessly to the mobile telecommunications device. The ECG electrode module may send information to a mobile telecommunications device that has been configured (e.g., by operating a program, applications (“app”), or the like) to receive and analyze information from the apparatus.

Electrocardiography is used to study the electrical activity of the heart, and may be used for both diagnosis and treatment. Electrocardiograms (ECG) can be recorded or taken using electrodes placed on the skin of the patient in multiple locations. The electrical signals recorded between electrode pairs are referred to as leads. Varying numbers of leads can be used to take the ECG, and different combinations of electrodes can be used to form the various leads. Examples of leads used for taking ECGs are 3, 5, and 12 leads. For a 12-lead ECG 10 electrodes are used with six on the chest and one on each of the patient's arms and legs.

There are different “standard” configurations for electrode placement that can be used to place the electrodes on the patient. For example, the arm and leg electrodes can be placed closer to the chest or closer to the extremity of the arm/leg. The varying placement of the electrodes on the arms and legs can affect the ECG and make it more difficult to compare to a standard ECG.

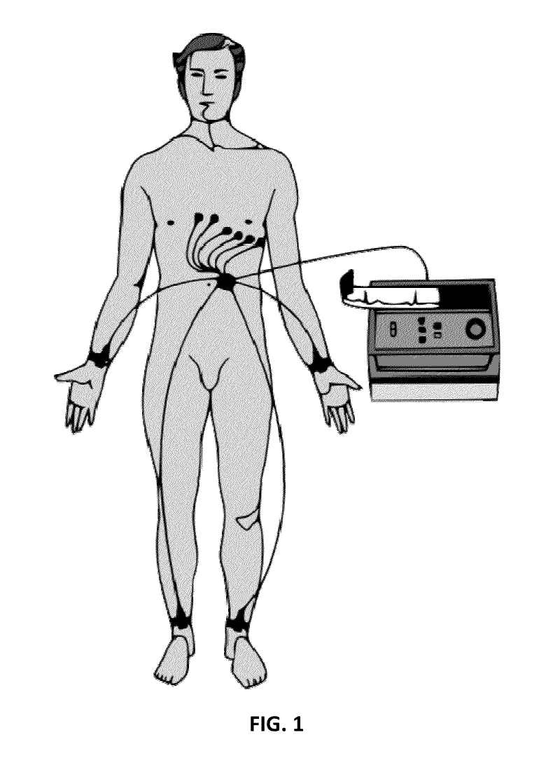

The standard or conventional 12-lead ECG configuration uses 10 electrodes. FIG. 1 illustrates a pictorial representation of the 10 electrodes, with 6 electrodes on the patient's chest and one electrode on each of the patient's arms and legs. The electrode placed on the right arm can be referred to as RA. The electrode placed on the left arm can be referred to as LA. The RA and LA electrodes are placed at the same location on the left and right arms, preferably near the wrist. The leg electrodes can be referred to as RL for the right leg and LL for the left leg. The RL and LL electrodes are placed on the same location for the left and right legs, preferably near the ankle.

Handheld ECG measurement devices are known, including devices that may adapt existing mobile telecommunications device (e.g., smartphones) so that they can be used to record ECS.

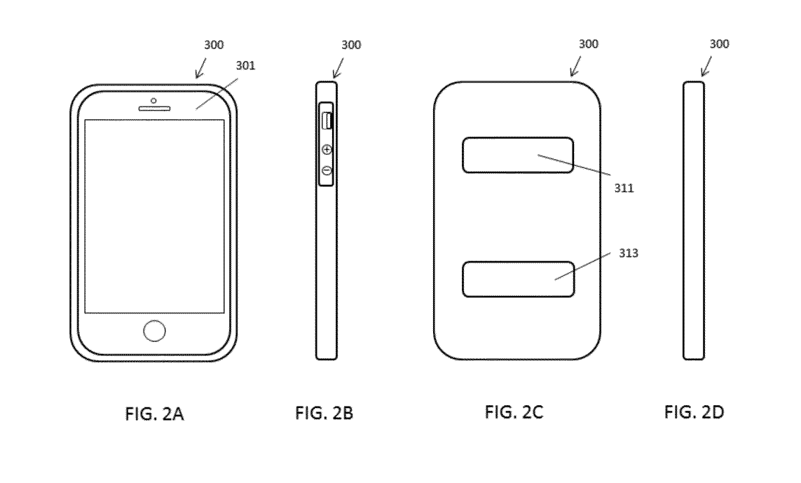

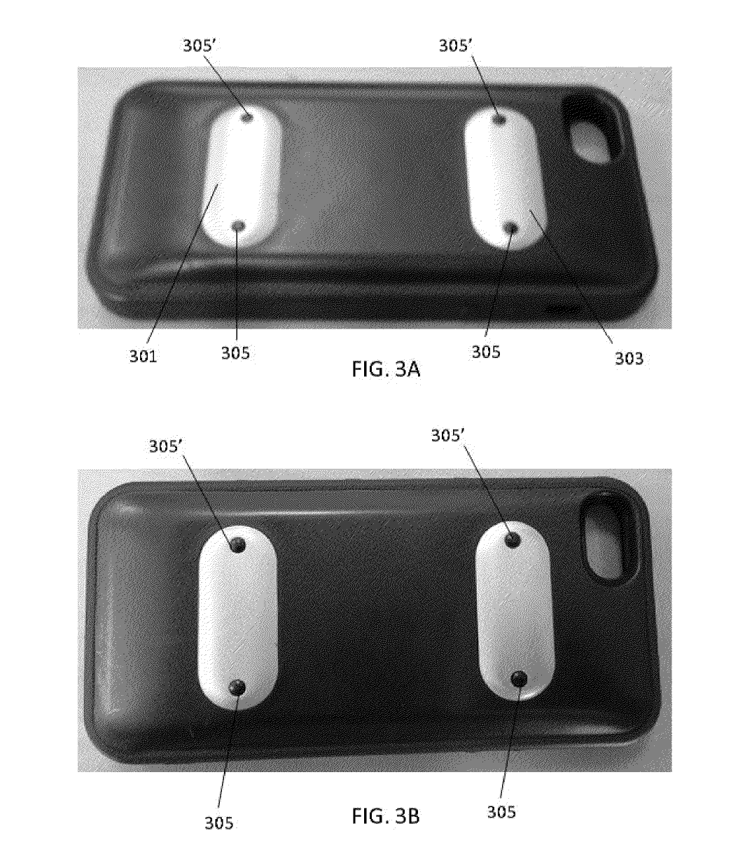

For example, FIGS. 2A-2D and 3A-3B illustrate one variation of a phone case including integrated electrodes that can record ECGs and communicate them to a mobile telecommunications device held within the case. Other examples of such devices are described, for example, in U.S. Pat. No. 8,509,882 and U.S. Pat. No. 8,301,232 (herein incorporated by reference in their entirely), as well as U.S. patent application Ser. No. 13/752,048, filed Jan. 28, 2013 and titled “ULTRASONIC TRANSMISSION OF SIGNALS,” Publication No. US-2013-0197320-A1; U.S. patent application Ser. No. 13/969,446, filed Aug. 16, 2013 and titled “ULTRASONIC TRANSMISSION OF SIGNALS;” U.S. patent application Ser. No. 13/964,490, filed Aug. 12, 2013 and titled “HEART MONITORING SYSTEM USABLE WITH A SMARTPHONE OR COMPUTER;” and U.S. patent application Ser. No. 13/108,738, filed May 16, 2011 and titled “WIRELESS, ULTRASONIC PERSONAL HEALTH MONITORING SYSTEM,” Publication No. US-2011-0301439-A1, each of which were previously incorporated by reference in their entirety.

However, because the form factor (e.g., shape, thickness, width, height, and locations of controls) of mobile telecommunications devices (e.g., smartphones) may vary between different manufacturers, and even different generations of devices, previously described ECG devices that are adapted for housing a mobile communications device such as a smart phone typically operate with only a single form factor, or limited variations of a single form factor, for the housing of the mobile telecommunications device. Thus, it would be beneficial to offer an ECG-sensing and transmitting apparatus for use with a mobile telecommunications device that can be used, or easily adapted for use, with a variety of mobile telecommunications devices, including mobile telecommunications devices having very different form factors. Such “universal” ECG-sensing and transmitting apparatuses for use with a mobile telecommunications devices of different shapes, sizes and/or configurations may be applied directly to a mobile telecommunications device, or may be used with a variety of differently-sized cases.

### ECG Electrode Module

Generally, an ECG electrode module (which may be referred to as an ECG module, electrode module, or simply “module”) includes two or more electrodes, or connectors for two or more electrodes, configured to be contacted by or to a patient/subject to sense an ECG signal from the patient/subject. A patient/subject may include any human or animal from whom the ECG is to be taken, referred to as either or both a “patient” or a “subject.” The ECG electrode module also typically includes a housing enclosing (or at least partially enclosing) control and/or communication circuitry for detecting, transmitting and/or storing ECG signals. In some variations the electrodes are on an outer surface of the housing of the ECG module, and may be sized and configured so that the can be contacted by the subject to detect an ECG (e.g., a single lead ECG). For example, the electrodes on the outer surface of the housing may be configured to be touched by a subject holding the device in their hand or hands; one electrode may be touched by a hand holding the device (e.g., a finger or fingers, thumb, etc.) and the second electrode may be touched by another portion of the body, including another hand, leg, chest, etc.

The control and/or communications circuitry may include hardware, software and/or firmware configured to detect, and in some variations amplify, filter, and/or smooth the electrical (e.g., ECG) signal sensed. The control circuitry may include a controller (e.g., microcontroller) configured to receive and process ECG signals from the electrodes.

In general the ECG electrode module may include communications circuitry and/or logic (including software and/or firmware) to process ECG signals for wireless transmission by the ECG electrode module to a mobile telecommunications device. Any appropriate wireless transmission modality may be used, including, but not limited to radio, sound (e.g., ultrasound), magnetic/electromagnetic, optical, etc. Ultrasound transmission may be particularly useful, as it may not require complex processing or a dedicated receiver, and may use the built-in audio receiver (microphone) in the mobile telecommunications device. Depending on the wireless transmission modality used, the electrical signal may be processed for transmission by a transmitter configured to operate in the particular wireless modality. The ECG electrode module may therefore include as part control and/or communications circuitry (and/or logic) a converter or converter assembly for converting detected ECG signals from the subject into a transmission signal for transmission by a transmitter that may also be included as part of the ECG electrode module. The signal transmitted can comprise for example any of a radio signal, a microwave signal a visible light signal, an infrared signal, a sonic signal, an ultrasonic signal, an electromagnetic induction signal, a WiFi signal, a ZigBee signal, a Bluetooth signal, a Bluetooth LE signal, or a wireless signal.

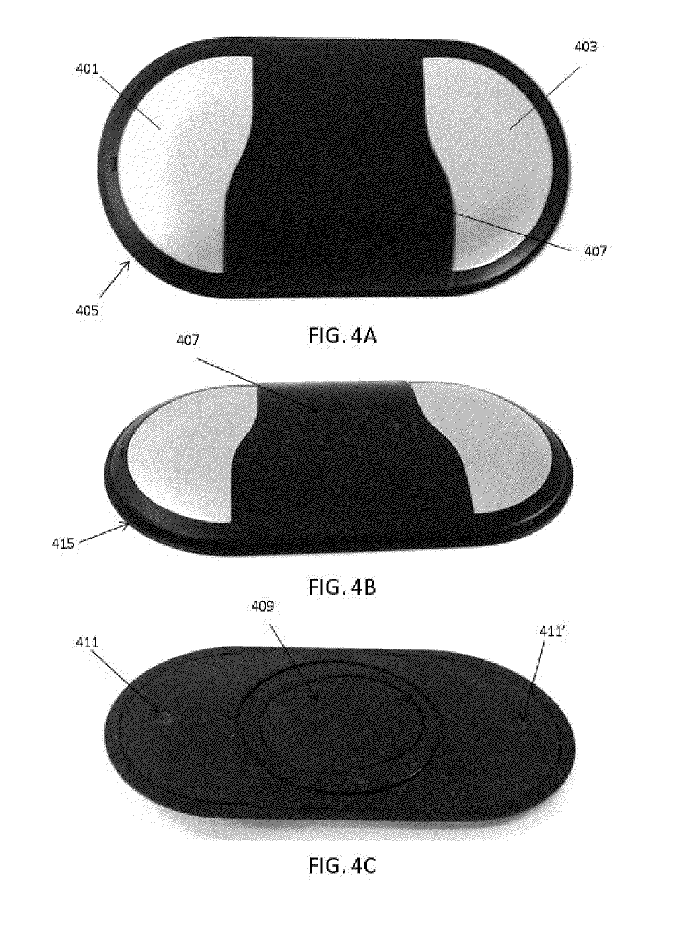

FIGS. 4A-4C shows one variation of a universal ECG electrode module in top, front perspective, and back perspective views, respectively. In FIG. 4A, the top view of the ECG module shows the generally oval shape of the ECG module. A housing 405 encloses the ECG module. A first electrode 401 is located on the left of the module on the top surface and a second electrode 403 is located on the right side of the top of top surface. Between the first and second electrodes, a portion 407 of the housing projects higher than the surfaces of the first and second electrodes, as can be seen in FIG. 4B.

In FIG. 4B, the portion of the housing between or around the first and second electrodes extends from the top of the ECG module higher than the first and second electrodes and forms a rest or resting surface or surfaces (or “foot”). This resting surface 407 shown in FIGS. 4A-4B is one variation of a resting surface that is configured so that when the ECG module (or a phone connected to the ECG module via a base unit) is placed top-side (or electrode-side) down on a table or other flat surface, the electrodes do not contact the table. This is unexpectedly and particularly useful at maintaining battery life, as in use, if the electrodes are placed on a wet or conductive surface (e.g., metal table), as is often found in a medical and home setting, the battery may be discharged by holding the device against the surface. A rest surface on the apparatus (e.g., on the ECG module and/or base unit) that prevents the electrodes from contacting the table surface may therefore prevent premature discharge of the battery or other power source of the ECG module. The rest surface may be non-conductive or may be separated from the electrodes by an insulator or non-conductive surface.

The concept of a rest surface that prevents the electrodes from resting against a table or flat surface when an apparatus is placed flat on a table may be applicable to other ECG detecting apparatuses, and is not limited to the ECG module variations. For example, FIGS. 3A and 3B show ECG devices similar to those described in U.S. application Ser. No. 13/752,048 and U.S. patent application Ser. No. 13/969,446, and other applications previously incorporated by reference in their entirety. In FIG. 3A, the electrodes (first 301 and second 303 electrodes) each include two small rest surfaces 305, 305′ (“bumps”) forming four feet on which the apparatus (holding a smartphone) will sit when placed electrode-side down on a table, preventing the electrodes from contacting the surface.

Returning to FIG. 4C, the back surface of the ECG electrode module shows the housing. The housing may include a door or cover 409 that may be opened to allow access to the inside of the housing, or a region of the inside of the housing, such as a battery compartment. This may allow the battery (or other internal components) to be switched out or changed. In FIG. 4C, the back region also includes two detent regions 411, 411′ than can engage with projections on a base unit (or vice-versa; the detents may be present on the base unit, or a detent and projection may both be on each). These detents maybe openings, concavities, pits, holes, etc., and may act as guides or positioning aids to help align and/or secure the ECG module in a base unit.

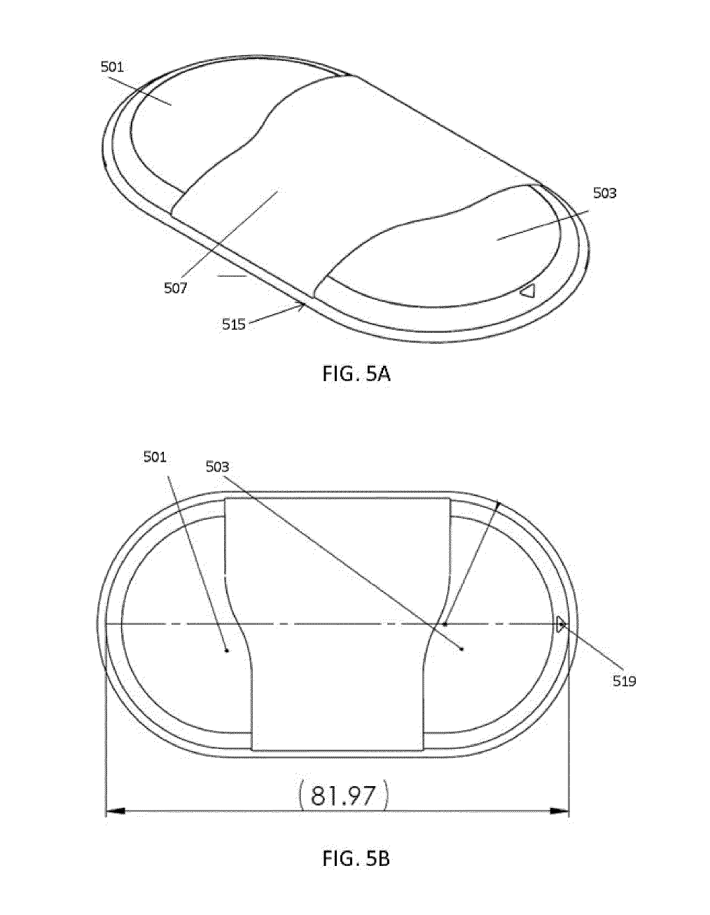

FIG. 5A shows another perspective view of an ECG module similar to the variation shown in FIGS. 4A-4C, and FIG. 5B shows a top view. In both FIGS. 4A-4C and 5A-5B, the ECG electrode module includes an outer rim or lip 415,515. As will be described below, this lip or rim region may extend completely or partially around the ECG module, and may help secure the module within the base unit. In FIGS. 5A and 5B the electrodes 501, 503 are again shown recessed relative to a portion of the upper housing that serves as a rest surface. The ECG module may also include an indicator 519 or reference mark that indicates the orientation (e.g., direction) and/or a status (on, off, transmitting, receiving, etc.) of the apparatus. The status indicator 519 may include an LED or other indicator.

In FIG. 5B, exemplary dimensions (showing a length of approximately 81.97 mm or 3.23 inches) are provided for illustration purposes only; other dimensions may be used. In general, the length of the ECG module may be between about 0.5 inches (13.00 mm) and about 6 inches (153.00 mm).

In the exemplary ECG module shown in FIG. 5B, the width of the device is approximately 40 mm (about 1.57 inches). In general, the width of the ECG module may be between about 0.5 inches (13.00 mm) and about 4 inches (101.6 mm) wide. The entire ECG electrode module may have a maximum thickness of 4.75 mm (i.e., about 0.19 inches); in some variations the thickness may be between about 1 mm and about 20 mm (i.e., between about 0.04 inches and about 0.80 inches). In some examples, the rest surface may be about 0.50 mm (0.02 inches) higher than the top surface of the electrodes.

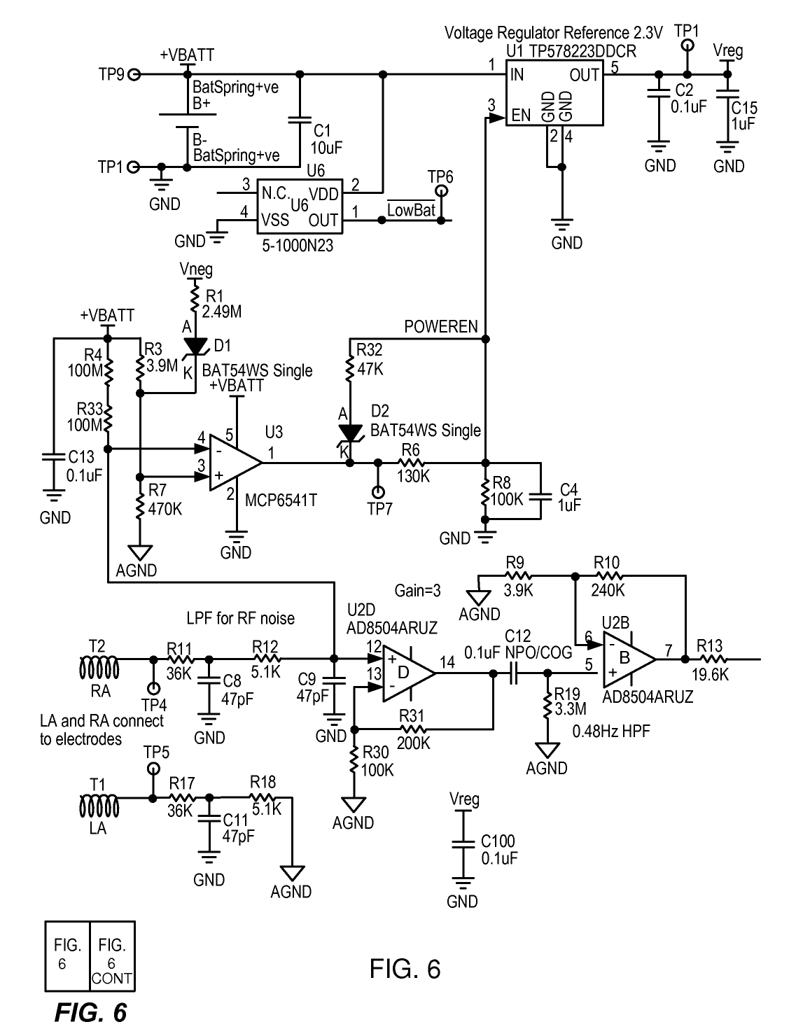

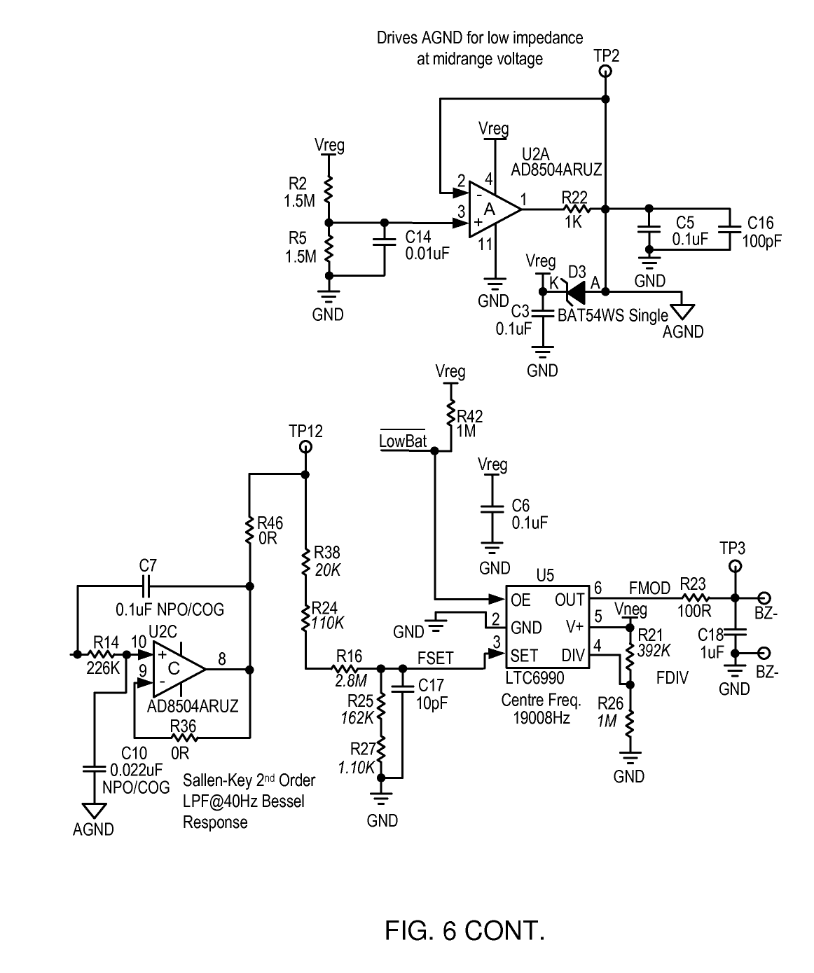

As mentioned, the housing of the ECG module may enclose (or partially enclose) the control and/or transmission sub-systems of the ECG electrode module. For example, the housing may at least partially enclose circuitry such as that shown in FIG. 6 for regulating the power source (battery), including amplifiers, filters and one or more microcontroller. The circuitry comprising filters, amplifiers and a microcontroller can for example regulate power sources, read ECG signals, process the ECG signals, and transmit signals in another format (e.g., ultrasound). The power regulation module may also absorb power shocks that will cause damage to the system. The reading module utilizes inductors, capacitors, resistors, filters and amplifiers to receive signals, remove noise, and extract desired signals. The transmission module receives the desired signals, based on which it resonates and generates ultrasound signals.

In FIG. 6, the controller is configured as a converter assembly to convert sensed electrical (ECG) signals into transmission signals configured to be transmitted wirelessly from the ECG module. Thus, the ECG module may for example transmit an ultrasound signal having a frequency of about 17 kHz or higher (e.g., between about 17 kHz and about 40 kHz, between about 17 kHz and about 30 kHz, between about 18 kHz and about 30 kHz, etc.).

In the exemplary ECG electrode modules described herein the module has a generally oval top profile that is slightly curved (e.g., at the edges). The ECG electrode module may have other profiles and shapes, including, but not limited to round, square, rectangular, etc. Virtually any shape may be used, as long as there is sufficient surface area for the electrodes (two or more electrodes) to make contact with the patient. The electrodes shown are symmetric and have equal surface areas. In some variations the electrodes may have different shapes and sizes.

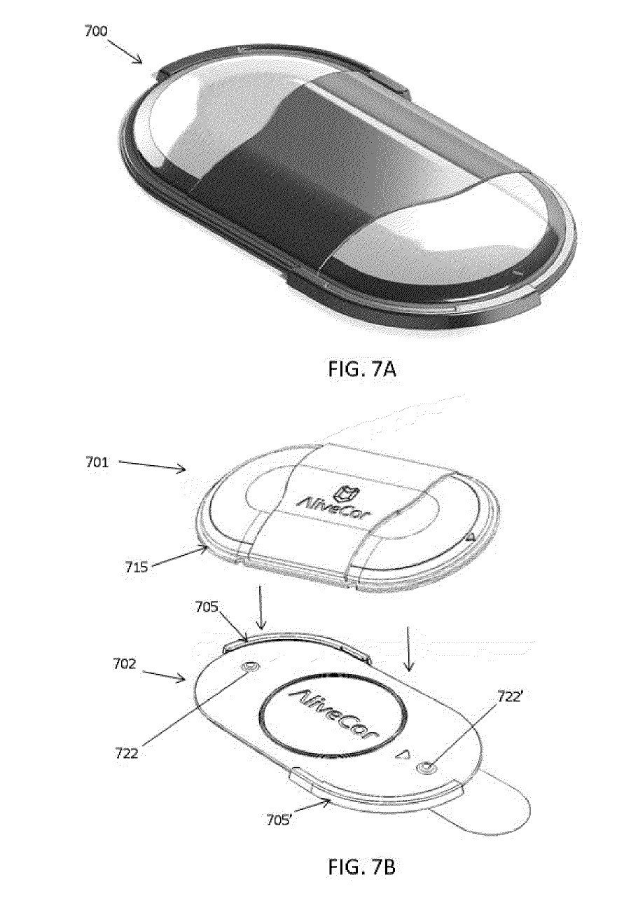

As mentioned, any of the ECG modules described may engage a base unit that can attach to a mobile telecommunications device such as a smartphone. FIG. 7B shows the variation of the ECG module 701 illustrated and described above engaging with a base unit 702. In this example, the ECG module 701 may be lowered onto the base unit 702, so that the ECG module fits between two retainers 705, 705′ on the edge of the base module. Once the module is held within the base unit, it may be rotated (e.g., clockwise in this example) so that the rim 715 of the ECG electrode module locks into the retainer lip region 705, 705′. Projections 722, 722′ on the base unit may snap into detents (not visible in FIG. 7B) on the bottom surface of the housing of the ECG module, confirming alignment and helping hold the device in position. The apparatus 700 formed by the combination of the base unit 702 and the ECG electrode module may be seen in FIG. 7A.

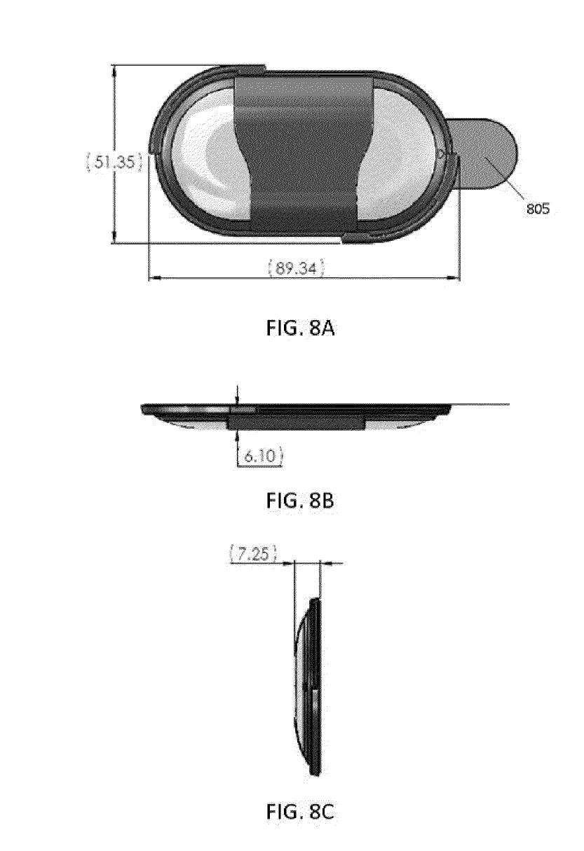

FIG. 8A shows a top view of another variation of an apparatus 800 including a base unit 802 into which an ECG electrode module 801 has been coupled. In FIG. 8A, the base unit 800 includes a tab 805 that is connected to a protective liner covering an adhesive backing on the base unit (not visible in FIG. 8A). FIG. 8A also shows exemplary dimensions in mm for the length and width of the combined apparatus (e.g., width of 51.35 mm and length of 89.34 mm). FIG. 8B shows a side view of the apparatus 800 of FIG. 8A, with the electrode surfaces facing downward. As can be seen in FIG. 8B, the housing of the ECG module 801 has a height that is greater than the height of the electrodes, so that the apparatus will rest on the housing (e.g., rest surface) and not the electrodes when placed on a flat surface such as a table. FIG. 8C shows a side view of the apparatus of FIGS. 8A and 8B.

In the example shown, the pull tab 805 may be removed to expose an adhesive back surface on the base unit. This adhesive back surface may be attached directly to a mobile telecommunications device (e.g., smartphone) or to a case for a mobile telecommunications device. Thereafter, the ECG electrode module may be removed from the base unit, but the base unit may stay attached to the smartphone or case. The same or a different ECG module may then be attached.

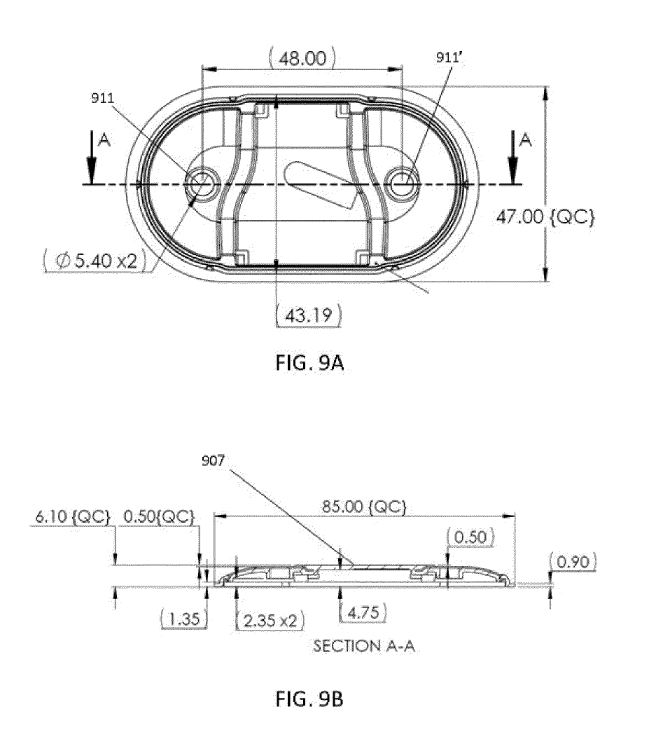

FIG. 9A shows a view of an ECG module 901 and base unit 902 coupled together. FIG. 9A also includes exemplary dimensions. In this example, the width of the base unit is approximately 47 mm (about 1.85 inches). In FIG. 9A, the detents 911, 911′ described in FIG. 4C are also shown and projections from the base unit are held within the detents. A section A-A of FIG. 9A is shown in FIG. 9B.

In FIG. 9B, a sectional view through the midline of the device of FIG. 9A shows an exemplary thickness of the entire ECG electrode module of about (at maximum) 6.10 mm (e.g., about 0.24 inches). The rest surface 907 is, for example, about 0.50 mm (0.02 inches) higher than the top surface of the electrodes.

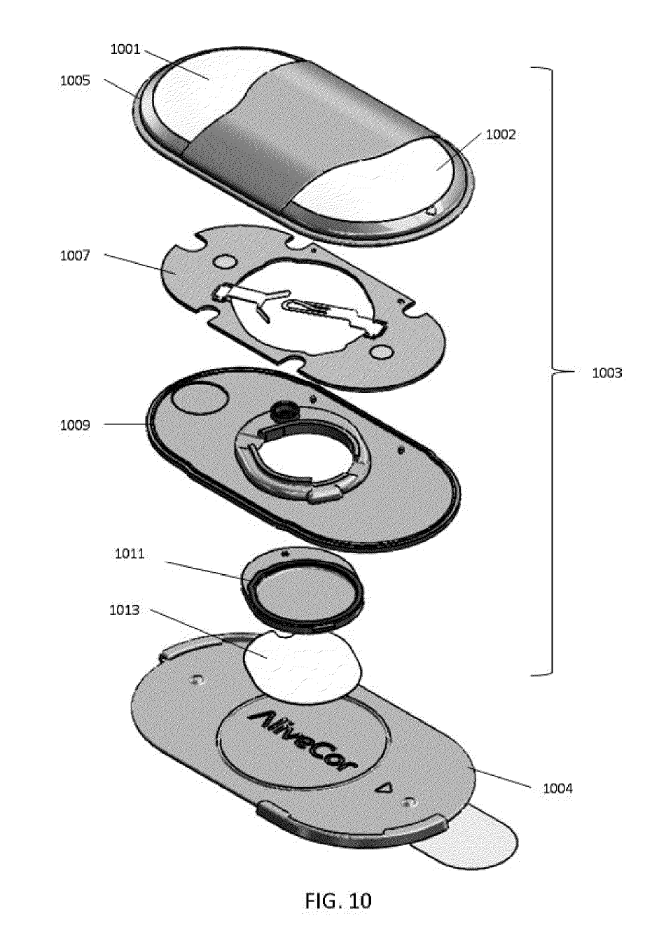

FIG. 10 shows an exploded view of an apparatus including an ECG module 1003 and a base unit 1004. In this example, components that may form an ECG module are also illustrated as part of the ECG electrode module 1003. For example, the ECG module includes a top portion of a housing 1005 (including first 1001 and second 1002 electrodes), an inner support structure 1007 for power/battery contacts (which may also support one or more PCBs and connections to the electrodes), a bottom portion of the housing 1009, and a battery holder 1011 and opening/door cover 1013.

As mentioned above, the base unit 1004 may be configured as a dock for the ECG module, as shown in FIG. 10, or it may include other structures and features. For example, in some variations, the base module is configured as a case or holder for holding a smartphone.

### Base Unit

In general, a base unit connects the ECG electrode module to a mobile telecommunications device. The base unit may connect to a mobile telecommunications device either temporarily (e.g., holding the mobile telecommunications device within a “case”), or permanently (e.g., adhesively). In general, the base unit may be sized and adapted to couple to a mobile telecommunications device. The base unit also typically includes a connection or interface region for securing and releasably holding an ECG electrode module. The connection or ECG electrode module interface region of the base unit may be a frictional engagement region that holds the ECG module by a friction fit and/or a snap, in which a portion of the base unit is temporarily displaced when engaging the ECG electrode module but ‘snaps’ or returns back to a preset position to hold the ECG module to the base.

As mentioned, a base unit may include a housing configured as a case for a mobile telecommunications device (e.g., smartphone). In variations in which the base is configured as a case to hold the mobile telecommunications device, the case may have an outer back surface and a front region through which a screen of the telecommunications device held in the case may be viewed.

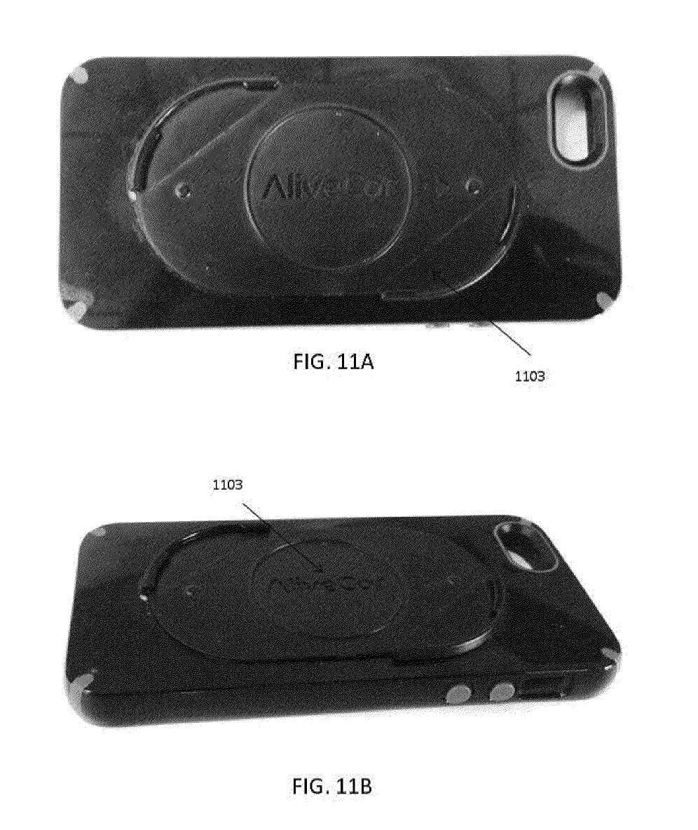

For example, FIGS. 11A and 11B show top and side perspective views, respectively, of a base unit configured as a case for a smartphone. In this example, the base unit is configured as a case for a smartphone and includes a smartphone holder or holding pocket with openings for one or more displays and/or controls of the smartphone, and also includes a mounting surface 1103 on the back of the base unit for releasably coupling with an ECG electrode module. In some variations this type of base unit may be formed by adhesively securing the dock base unit 1004 shown in FIG. 10 to any appropriate smartphone case (e.g., a case configured to enclose a smartphone having an appropriate form factor). In some variations the docking region (mounting surface) may be integrally formed with the case.

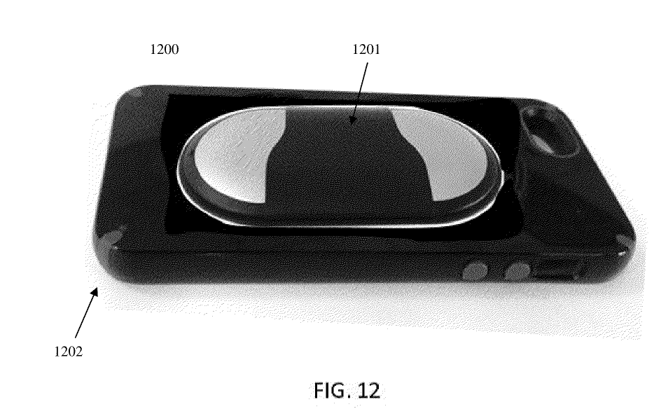

FIG. 12 illustrates one variation of a base unit 1202 configured as a case for a smartphone to which an ECG electrode module has been coupled. In general, the ECG module 1201 may be coupled to the base unit 1201 and the overall apparatus 1200 including the base unit 1202 and ECG electrode module 1201 may be connect to a smartphone (or other mobile telecommunications device) without interfering with the function of one or more controls/output/inputs of the smartphone.

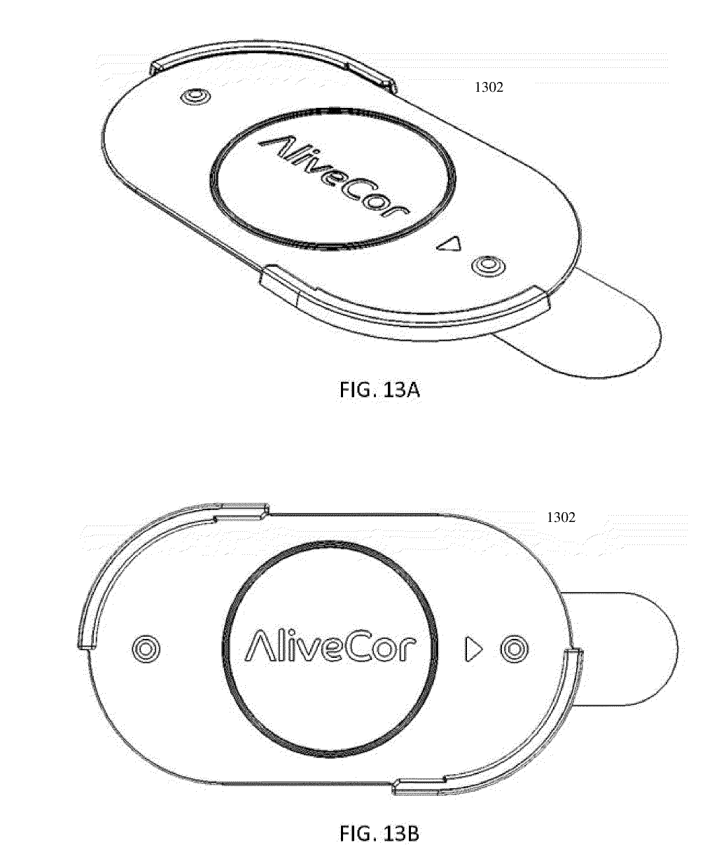

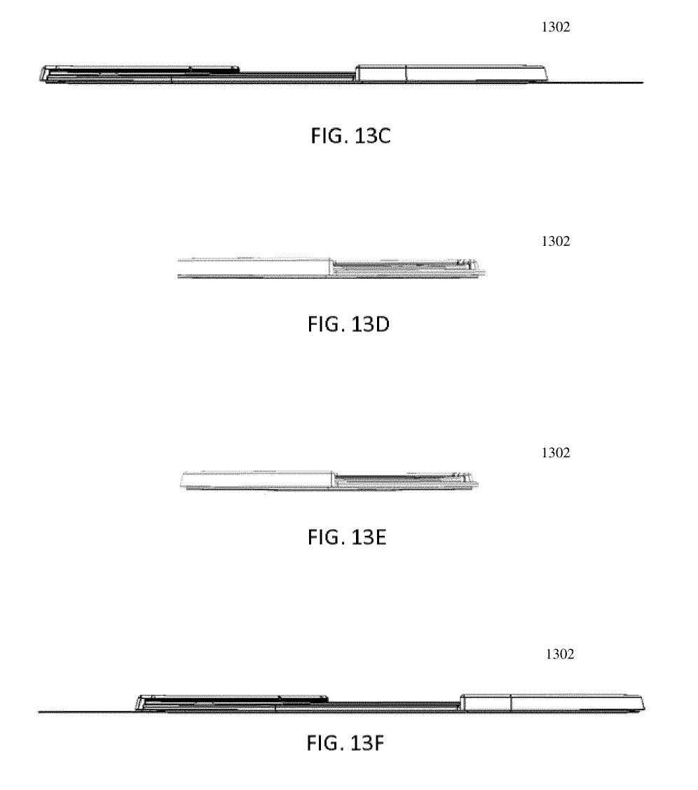

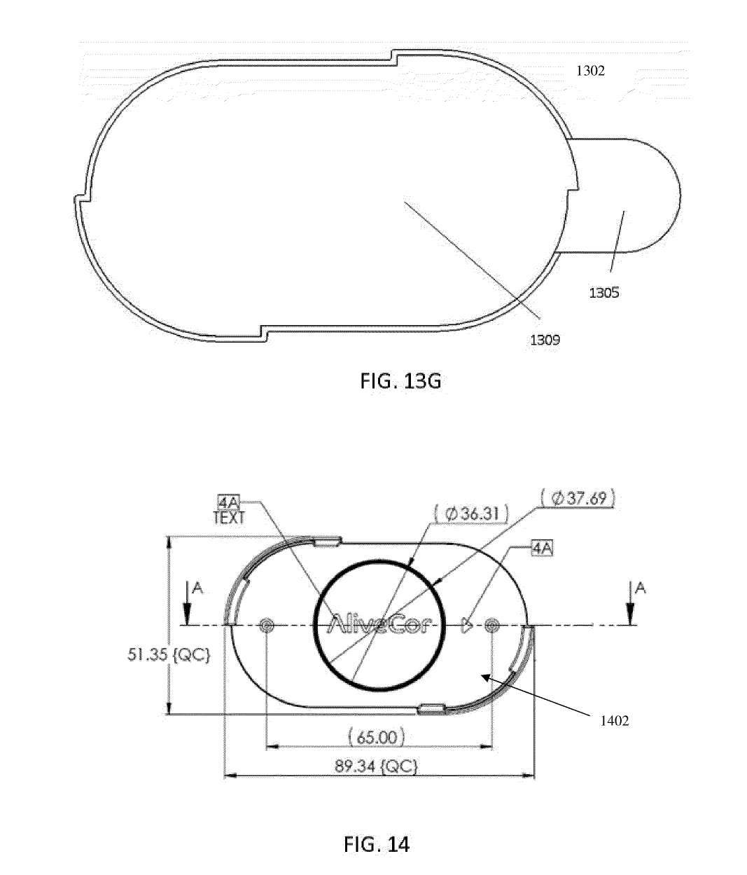

FIGS. 13A-13G show various views of one example of a base unit 1302 that may releasably secure and couple with an ECG electrode module. In this example, the base unit is primarily the docking region of the base unit, but includes an adhesive backing (visible in FIG. 13G) that can be exposed by removing a protective cover 1309 (e.g., by pulling a tab 1305). As mentioned, the base unit 1302 can then be mounted to a mobile telecommunications device or to a case/cover for mobile telecommunications device.

FIG. 14 shows another example of a base unit 1402 with exemplary dimensions indicated for illustrative purposes only.

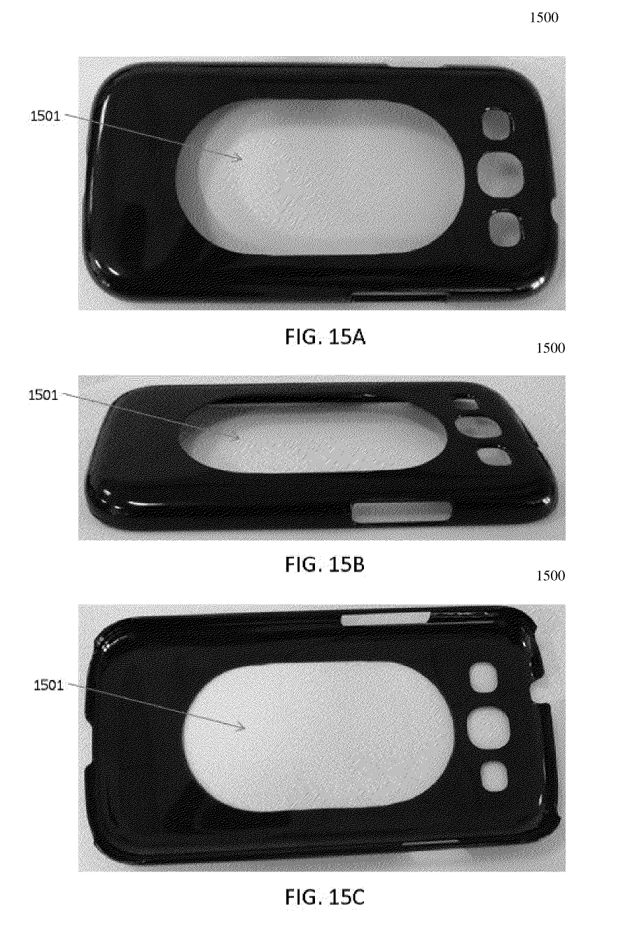

Some base units include an opening into which an ECG electrode module may fit in order to releasably secure the ECG module to a mobile telecommunications device. For example, FIGS. 15A-15C illustrate one variation of a base unit 1500 configured as a smartphone case having an opening 1501 into which an ECG electrode module may fit. The diameter of the opening 1501 may generally be configured to be slightly less than the outer diameter of the ECG module, in particular, the outer rim (e.g., 515 in FIG. 5A) of the ECG module may have a slightly greater diameter than the opening, so that the edge of the opening mates with this outer rim region of the ECG module; when the ECG module is inserted into the back (inside the pocket/case configured to hold the smartphone) of the base unit, the ECG module is retained within the opening. Inserting a smartphone into the case (base unit) may further secure the ECG module against the base unit.

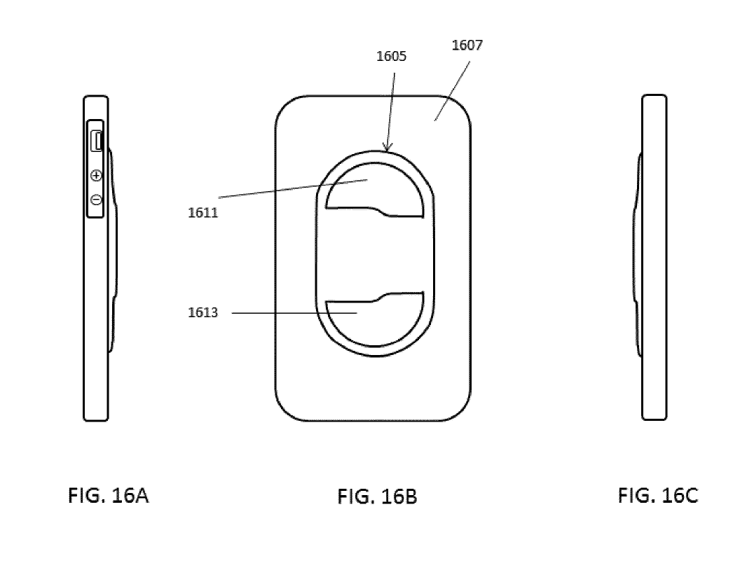

FIGS. 16A-16C illustrate one example of an apparatus 1600 including an ECG electrode module 1605 and a base unit 1607 (configured as a case) connected to a smartphone such as an iPhone™. In FIG. 16A, the ECG module 1605 includes two electrodes 1611, 1613. In use, the device 1600 may be held by a subject who may initially place the smartphone in a ready state to receive a transmitted ECG signal. For example, the user may activate an application software program (logic) so that the smartphone processor examines a receiver (e.g., the microphone) for wirelessly transmitted signals (e.g., ultrasound signals). The subject may then touch each of the two electrodes to generate an ECG signal. For example, a lead I (LA-RA) recording may be taken when the subject's right hand touches the first electrode and the subject's left hand touches the second electrode. Other leads may be recorded (guided by the smartphone software), e.g., by contacting other region of the body (right leg, left leg, chest, etc.).

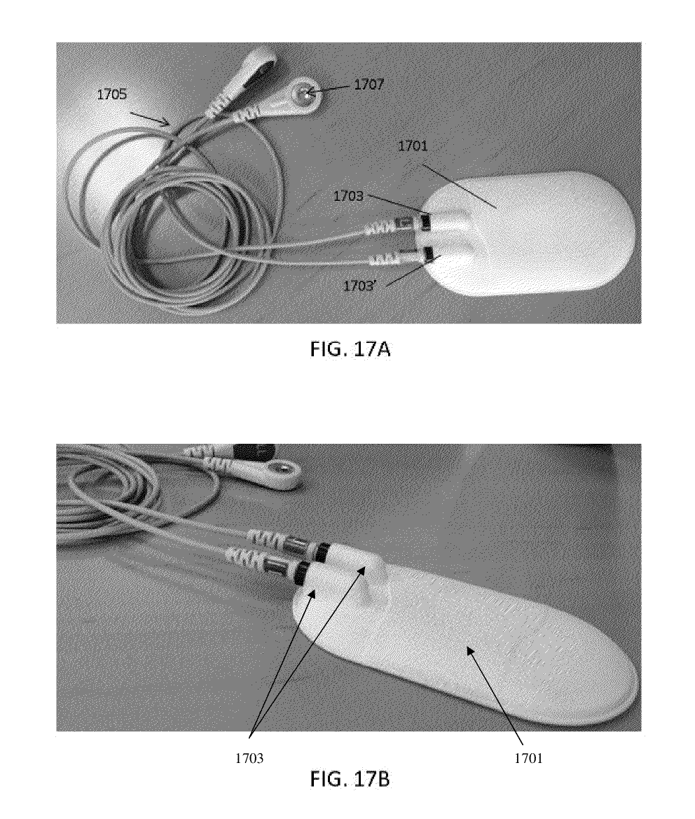

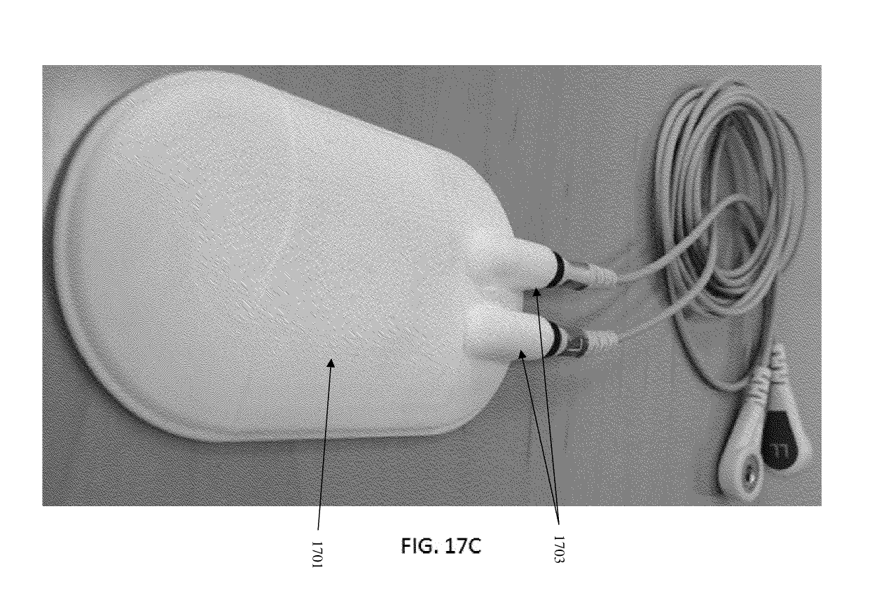

In some variations the ECG electrode module includes ECG electrodes that are configured to connect to the module by a wire. For example, FIGS. 17A-17C show views of an ECG electrode module 1701 having a pair of connectors 1703, 1703′ for connecting to traditional ECG electrodes which include cables/wires 1705 including the ECG contact 1707 and a plug connecting to the ECG electrode module. Any appropriate electrode (e.g., including pad, conductive adhesive/gel, etc.) may be used; in particular, the electrodes may be plugged/unplugged into the ECG electrode module as desired.

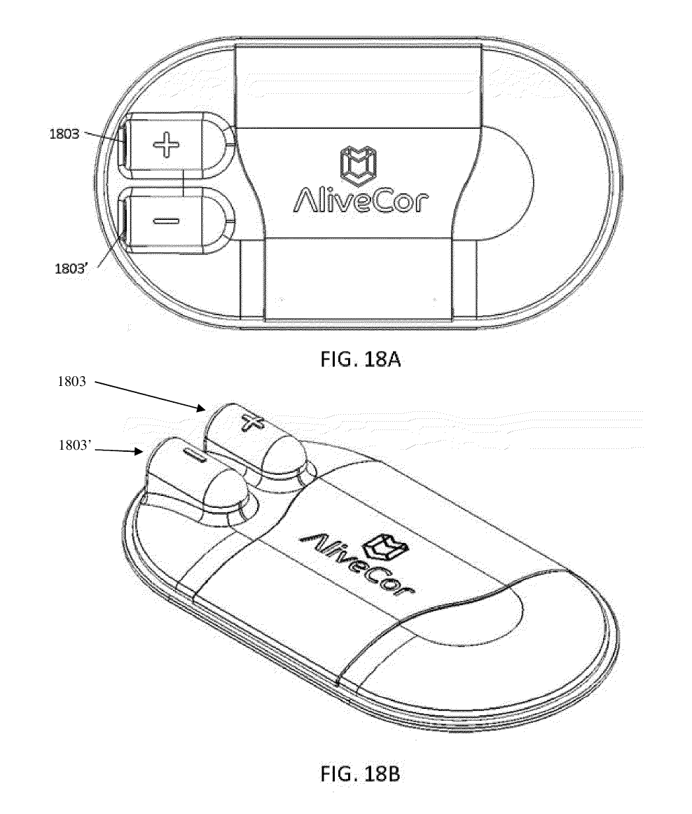

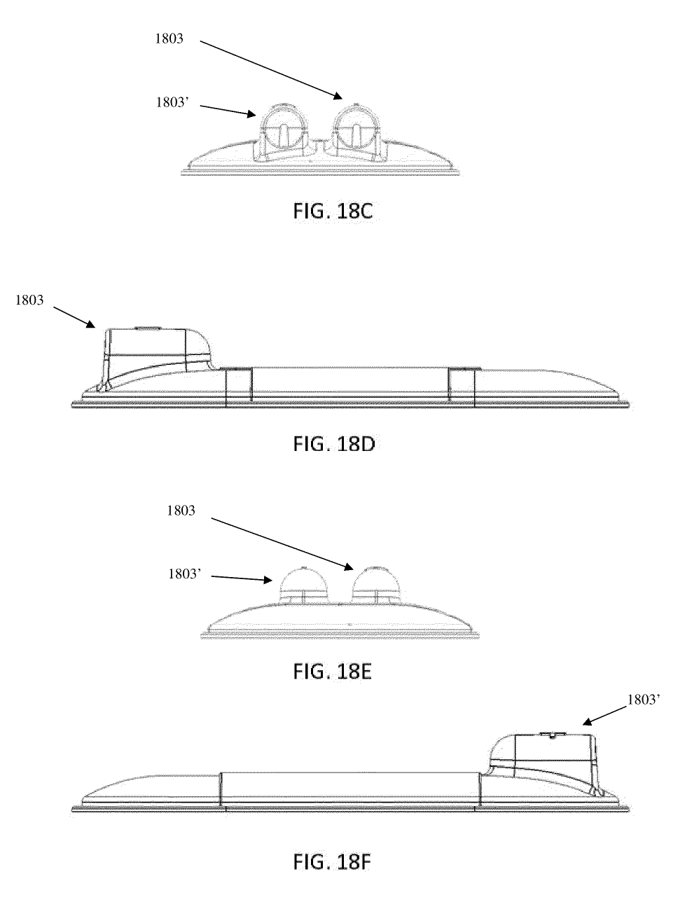

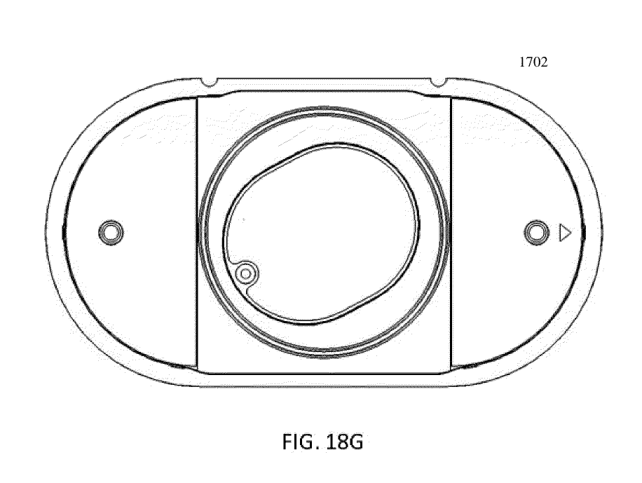

FIGS. 18A-18G shows various views of the ECG electrode module 1701 of FIGS. 17A-17C, in which the ECG module 1701 includes connectors 1803, 1803′ for electrodes. Thus, in any of the variations of the ECG electrode modules described herein the ECG electrode module may include electrode connectors 1803, 1803′ instead of, or in addition to, electrodes. In this example, the connectors 1803, 1803′ include sockets into which the electrode cables plug. Any appropriate connector (socket, plug, prong, clip, etc.) may be used. The apparatus 1701 may otherwise be operated the same as described above. For example, the ECG electrode module 1701 including electrode connectors 1803, 1803′ may be coupled (e.g., removably coupled) with a base unit 1702 and connected to a mobile telecommunications device, and may wirelessly communicate with the mobile telecommunications device.

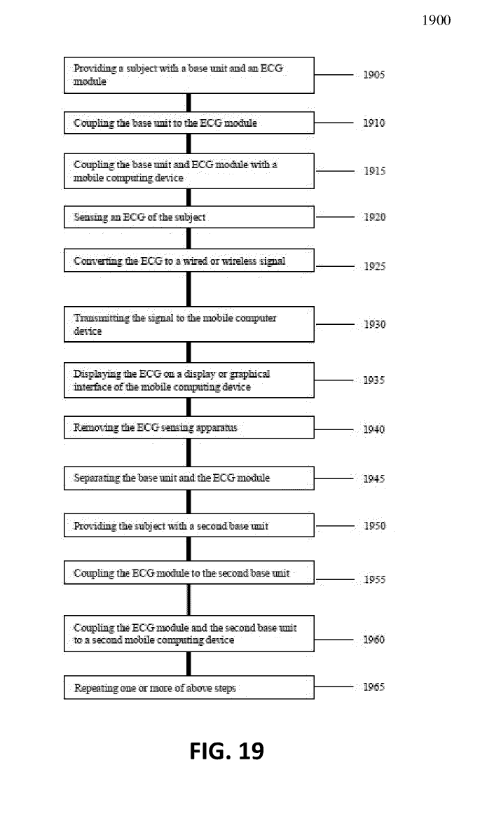

FIG. 19 shows a method 1900 for sensing an ECG of a subject. In a step 1905, a subject is provided with a first base unit and an ECG module. The first base unit may comprise any of the base units described herein. The ECG module may comprise any of the ECG modules described herein.

In a step 1910, the first base unit may be coupled to the ECG module to form a first ECG sensing apparatus. The subject may be instructed to couple the first base unit with the ECG module with a series of sub-steps as can be understood from the present disclosure.

In a step 1915, the first ECG sensing apparatus may be coupled to a first mobile computing device. The subject may be instructed to couple the first ECG sensing apparatus to the first mobile computing device with a series of sub-steps as can be understood from the present disclosure. It should be understood that the base unit can be coupled with the ECG module either before or after the base unit is coupled to the first mobile computing device.

In a step 1920, the first mobile computing device coupled with the first ECG sensing device may be used to sense an ECG of the subject. For example, the ECG module may comprise first and second sensor electrodes that the subject may contact with his or her skin by manipulating the sensing apparatus enclosed mobile computing device. The contact may be with the skin of the subject's chest as described herein. For example, the contact may be with skin of first and second limbs of the subject (where the skin may be for example of the right arm and left arm for a Lead I ECG, the left leg and right arm for a Lead II ECG, and the left leg and left arm for a Lead III ECG).

In a step 1925, the sensed ECG may be converted to a wired or wireless signal by the ECG module.

In a step 1930, the signal may be transmitted to the mobile computing device. For example, the signal may be transmitted wirelessly as described herein. Alternatively or in combination, the signal may be transmitted with a wired connection such as a USB connection, a Lightning connection, or an audio port connection.

In a step 1935, the ECG may be displayed by a display and/or graphical user interface of the computing device. The computing device may store the ECG, generate a diagnosis or analysis of the ECG to provide to the subject, and/or send the ECG and/or diagnosis or analysis to a remote server such as through the Internet.

In a step 1940, the first ECG sensing apparatus may be removed from the computing device. The subject may be instructed to remove the first ECG sensing apparatus from the mobile computing device with a series of sub-steps as can be understood from the present disclosure.

In a step 1945, the first base unit and the ECG module may be separated. The subject may be instructed to separate the first base unit from the ECG module with a series of sub-steps as can be understood from the present disclosure. It should be understood that the base unit can be separated from the ECG module either before or after the base unit is removed from the mobile computing device.

In a step 1950, the subject may be provided with a second base unit which may have a form factor different than the first base unit.

In a step 1955, the ECG module may be removably coupled to the second base unit to form a second ECG sensing apparatus. The subject may be instructed to couple the second base unit with the ECG module with a series of sub-steps as can be understood from the present disclosure.

In a step 1960, the second ECG sensing apparatus may be coupled to a second mobile computing device different from the first mobile computing device. For example, the first ECG sensing apparatus may not fit with the second mobile computing device and vice versa. The subject may be instructed to couple the second ECG sensing apparatus to the second mobile computing device with a series of sub-steps as can be understood from the present disclosure. Accordingly, the same ECG sensing module may be used with different mobile computing devices with different form factors. A subject switching or upgrading their smartphone, for instance, may need only acquire a base unit suitable for the newer smartphone while retaining the ECG module.

In a step 1965, one or more of the above ECG sensing and processing steps may be repeated.

Although the above steps show the method 1900 of sensing an ECG signal in accordance with many embodiments, a person of ordinary skill in the art will recognize many variations based on the teaching described herein. The steps may be performed in different orders. Steps may be added or omitted. Some of the steps may comprise sub-steps. Many of the steps may be repeated as often as desirable.

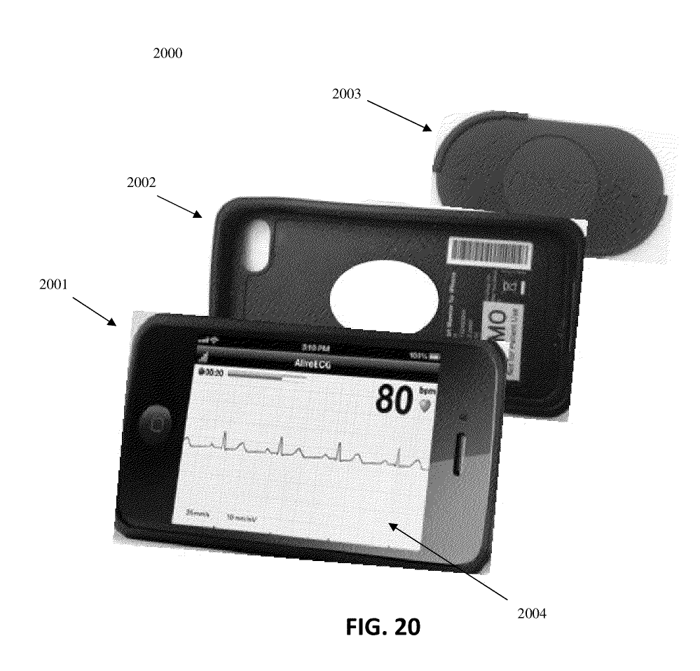

FIG. 20 shows an example of an exploded view of a variation of an ECG sensing system 2000. A mobile computing device 2001 has a display 2004, which displays an ECG wave. A base unit 2002 can removably attach to the mobile computing device 2001. An ECG module 2003 can removably attach to the base unit 2002. In this example the mobile computing device 2001 is a smartphone but it is understood that the mobile computing device 2001 can also for example comprise tablet computers, smart watches, or wearable computing devices.

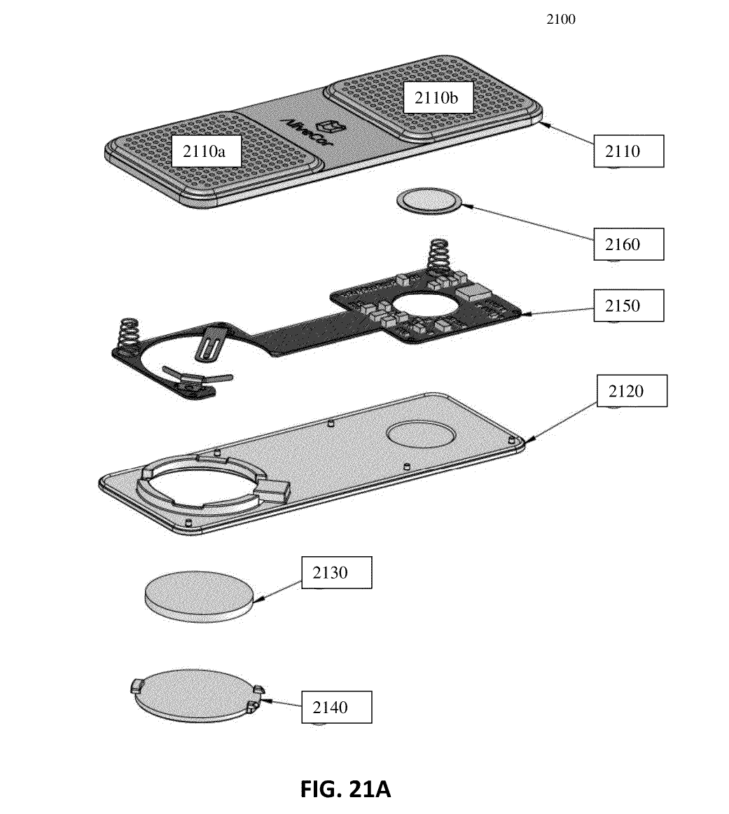

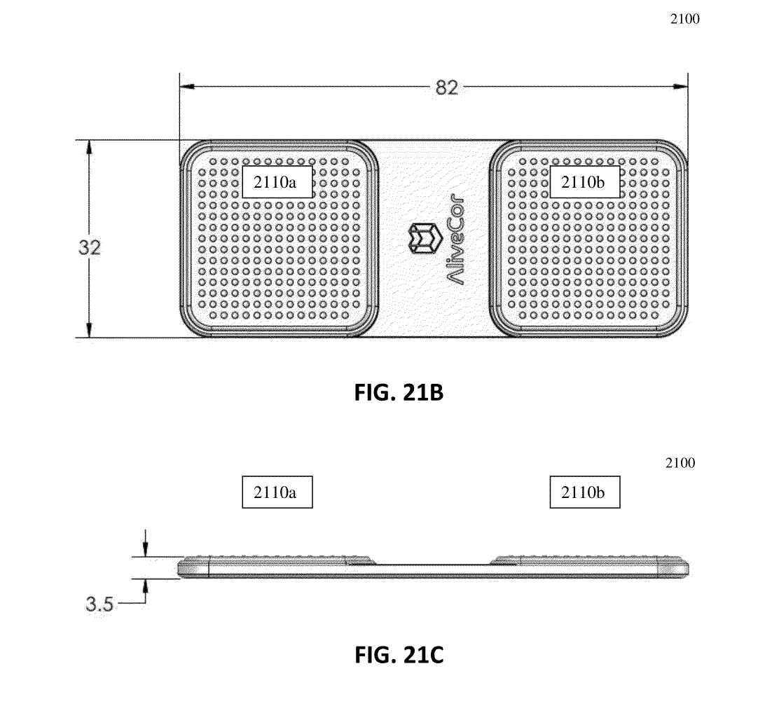

FIG. 21A shows an exploded view of a variation of the biosensor or ECG module comprising a slim biosensor ECG module 2100. FIGS. 21B and 21C show top and side views, respectively of the slim biosensor ECG module 2100. The slim ECG module 2100 may comprise an upper housing 2110, a lower housing 2120, a power supply or battery 2130, a power supply or batter cover 2140, sensor circuitry 2150, and a transmitter element 2160. The upper housing 2110 may comprise a first sensor electrode 2110a and a second sensor electrode 2110b. The sensor circuitry 2150 may be housed within the upper housing 2110 and the lower housing 2120 along with the power supply or battery 2130 and the transmitter element 2160. The sensor circuitry 2150 may comprise a printed circuit board configured to measure and process one or more biosignals such as an electrocardiogram. The sensor circuitry 2150 may be coupled to the first and second sensor electrodes 2110a, 2110b to intake a biosignal reading. The sensor circuitry 2150 may be coupled to the transmitter element 2160 to transmit the biosignal reading to another device. The transmitter element 2160 may comprise a piezoelectric transmitter to transmit the biosignal reading with an audio or sound signal, such as an ultrasound signal as described herein. Other methodologies of wired or wireless transmission may be used alternatively or in combination. The sensor circuitry 2150 and the transmitter element 2160 may be coupled to the power supply or battery 2130 to power the sensor circuitry 2150 and the transmitter element 2160. The outside dimensions of the shown exemplary slim ECG module 2100 can be, for example, about 32 mm in width, 82 mm in length, and 3.5 mm in height. When coupled to another computing device such as a smartphone or a tablet computer as described herein such as with use of a universal base unit, the slim biosensor or ECG module 2100 provides little obstruction to the normal use of the host computing device.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1 of the stated value (or range of values), +/−1 of the stated value (or range of values), +/−2 of the stated value (or range of values), +/−5 of the stated value (or range of values), +/−10 of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An electrocardiogram (ECG) sensing device for use with a mobile computing device, said ECG sensing device comprising
a base unit;
a universal coupler; and
an ECG module removably and universally couplable to said base unit via said universal coupler.

2. The device of claim 1, wherein said ECG module comprises a conductive contact surface, wherein said ECG module generates a signal in response to said conductive contact surface contacting a skin surface of a subject, and a wireless transmitter for wirelessly transmitting said signal to said computing device.

3. The device of claim 2, wherein said signal is one or more of a radio signal, a microwave signal, visible light signal, an infrared signal, a sonic signal, an ultrasonic signal, an electromagnetic induction signal, a WiFi signal, a ZigBee signal, a Bluetooth signal, a Bluetooth LE signal, or a wireless signal.

4. The device of claim 1, wherein said universal coupler comprises an opening through the base unit sized such that said ECG module may extend partially therethrough, said opening having an edge region configured to engage a periphery of said ECG module.

5. The device of claim 1, wherein said mobile computing device comprises a smartphone.

6. The device of claim 1, wherein said mobile computing device comprises a tablet computer.

7. The device of claim 1, wherein said mobile computing device comprises a smart watch.

8. The device of claim 1, wherein said mobile computing device comprises a wearable computer.

9. An ECG sensing system comprising
a mobile computing device; and
an ECG module comprising
a conductive contact surface for contacting a skin surface of a subject; and
an elevated portion having a height that projects above the height of said conductive contact surface.

10. The system of claim 9, wherein said conductive contact surface generates a signal in response to being at least partially contacted by a skin surface of a subject.

11. The system of claim 10, comprising a transmitter for wirelessly transmitting said signal to the mobile computing device.

12. The system of claim 11, wherein said signal is one or more of a radio signal, a microwave signal, visible light signal, an infrared signal, a sonic signal, an ultrasonic signal, an electromagnetic induction signal, a WiFi signal, a ZigBee signal, a Bluetooth signal, a Bluetooth LE signal, or a wireless signal.

13. The system of claim 12, wherein said mobile computing device comprises a display and a wireless receiver configured to receive the signal transmitted by said ECG module, said transmitted signal comprising an ECG.

14. The system of claim 13, wherein said display comprises a graphic or text representation of said ECG.

15. The device of claim 9, wherein said mobile computing device comprises a smartphone.

16. The device of claim 9, wherein said mobile computing device comprises a tablet computer.

17. The device of claim 9, wherein said mobile computing device comprises a smart watch.

18. The device of claim 9, wherein said mobile computing device comprises a wearable computer.
